(19) United States
(12) Patent Application Publication
(10) Pub. No.: US 2015/0204881 A1
(43) Pub. Date: Jul. 23, 2015

(54) Biomarkers For Diagnosis Of Diabetes And Monitoring Of Anti-Diabetic Therapy

(71) Applicant: Sumitomo Bakelite Co., Ltd., Tokyo (JP)

(72) Inventors: Hidehisa Asada, Washington Township, NJ (US); Diane Mccarthy, Warrington, PA (US); Yoshiaki Miura, Glen Rock, NJ (US); Taku Nakahara, West New York, NJ (US)

(21) Appl. No.: 14/395,132

(22) PCT Filed: Apr. 19, 2013
(86) PCT No.: PCT/US2013/037437

§ 371 Date: Oct. 17, 2014

Related U.S. Application Data
(60) Provisional application No. 61/635,911, filed on Apr. 20, 2012.

(51) Int. Cl. G01N 33/66; G01N 27/447

(52) U.S. Cl. CPC G01N 33/66; G01N 27/44791; G01N 2400/38; G01N 2800/52; G01N 2800/042

(57) ABSTRACT

The present invention relates to the use of N-linked glycan profiles of blood or blood component proteins as biomarkers for diagnosing diabetes mellitus and for monitoring the efficacy of anti-diabetic therapy. Specifically, the present invention relates to detecting changes in the amounts of N-linked glycans as diagnostic biomarkers for diabetes mellitus and as indicators of the efficacy of anti-diabetic therapy over time.

This is the National Phase entry of International Patent Application No. PCT/US2013/037437, filed Apr. 19, 2013, which claims the benefit of U.S. Provisional Application No. 61/635,911, filed Apr. 20, 2012, both of which are incorporated by reference herein in their entireties.

### TECHNICAL FIELD

The present invention relates to the use of N-linked glycan profiles of blood and blood component proteins as biomarkers for diagnosing diabetes mellitus and for monitoring the efficacy of anti-diabetic therapy. Specifically, the present invention relates to detecting changes in the amounts of N-linked glycans as diagnostic biomarkers for diabetes mellitus and as indicators of the efficacy of anti-diabetic therapy over time.

### BACKGROUND

The major biochemical alteration in type 2 diabetes is hyperglycemia, which is typically caused by a combination of impaired insulin secretion from pancreatic β-cells and insulin resistance in peripheral tissues. Hyperglycemia is also a causative factor in the development of micro- and macrovascular complications in diabetic patients. For these reasons, maintaining blood glucose levels within the normal range (glycemic control) is a primary goal of anti-diabetic therapy and on-going monitoring of blood glucose during anti-diabetic therapy, either directly or via detection of a correlated biomarker, is necessary for this purpose. There is continuing interest in development of glucose monitoring methods and hypoglycemic anti-diabetic drugs which can provide improved levels of glycemic control. In the research setting, the db/db mouse is an accepted animal model for human type 2 diabetes. The db/db mouse is characterized by a G-to-T point mutation of the leptin receptor gene, which results in abnormal receptor splicing and defective leptin signaling. These mice exhibit many of the metabolic abnormalities of human type 2 diabetes, including hyperglycemia, obesity and early hyperinsulinemia with subsequent renal pathologies. In the db/db mouse, improved glycemic control results in reduction in both blood glucose levels and levels of the glycated hemoglobin HbA1c.

Quantitation of glycated (glycosylated) hemoglobins is currently accepted as a relevant indicator or biomarker of long-term blood glucose control in patients with diabetes mellitus. Glycated hemoglobin is a relatively stable condensation product of hemoglobin and glucose (and possibly glucose phosphates), in contrast to the more labile hemoglobin-glucose adducts, which are believed to be of the aldimine (Schiff base) type generated by a non-enzymatic reaction between glucose and amino groups of hemoglobin. The hemoglobin-glucose adducts are believed to be converted into the stable glycated hemoglobin form via an Amadori rearrangement (cf. M. Roth: Clin. Chem. 29 (1983) 1991).

Glycated hemoglobin A was recognized when hemoglobin A was subjected to electrophoresis and cation exchange chromatography. Owing to the more negative charge and consequently higher electrophoretic migration rate towards the anode than that of the major component hemoglobin A (HbAo), glycated hemoglobin A was identified as “fast” hemoglobin (HbA1). HbA1 comprises a series of minor hemoglobins, including HbA1a, HbA1b and HbA1c, which are identified according to their different migration rates. Of these, HbA1c is present in greatest quantity in erythrocytes both from normal subjects and from diabetic patients. HbA1c is known to be glycated at the N-terminal valine of the beta-chains of hemoglobin A. However, recent studies have indicated that glycation may also occur at the amino group of lysine side chains and that all hemoglobins, including HbAo and HbA1c, may comprise such glycated sites. The labile (aldimine) precursor of HbA1c (usually referred to as “pre-HbA1c”) is not encompassed by the above definition of HbA1c.

It is now generally accepted that the level of HbA1c in a blood sample is a good index for the individual's glycemic control. Normal adults have about 90 percent of their total hemoglobin A as HbAo and 3-6 percent as HbA1c, the balance consisting of other minor hemoglobins including HbA1a and HbA1b. However, the level of HbA1c in patients with type 1 (juvenile) and type 2 (maturity-onset) diabetes ranges from about 6 percent to about 15 percent. The quantification of the HbA1c level in diabetic patients is regarded as a useful means of assessing the adequacy of diabetes control, in that such measurements represent time-averaged values for blood glucose over the preceding 2-4 months (cf. J. S. Schwartz et al.: Annals of Intern. Med. 101 (1984) 710-713). However, changes in HbA1c levels are somewhat delayed in response to an efficacious anti-diabetic therapy or treatment, due to the stability of the glycated form. Therefore, there remains a need for methods and biomarkers useful for diagnosing diabetes or pre-diabetes, and for monitoring glycemic control in response to anti-diabetic therapy that precede or predict the subsequent changes in HbA1c.

### SUMMARY OF THE INVENTION

The present invention provides N-linked glycan biomarkers associated with blood or blood component proteins and their use for diagnosing diabetes or pre-diabetes, and for evaluating the efficacy of anti-diabetic therapy over time. In a particular aspect, the present invention utilizes monitoring of the changes in the N-glycan composition of blood and blood component proteins over time during intervention therapy for diabetes for determination of glycemic control and evaluation of the efficacy of the anti-diabetic therapy. In another aspect, the present invention provides methods for diagnosis of diabetes or pre-diabetes utilizing quantitation of the N-glycans of blood and blood component proteins in a subject as compared to normal amounts of the corresponding blood and blood component protein N-glycans in normoglycemic blood or blood components. In a further aspect, the present invention provides methods for diagnosis of diabetes or pre-diabetes in a subject utilizing quantitation of the N-glycans of blood and blood component proteins in a subject as compared to amounts of the corresponding N-glycans in blood or blood components in the subject prior to development of diabetes or pre-diabetes.

It has been discovered that the N-linked glycosylation pattern or composition of blood or blood component proteins (or total N-glycan composition) of a diabetic individual or patient changes over time in response to an anti-diabetic intervention therapy. The change in N-linked glycosylation pattern or composition of total blood or blood component protein (or total N-glycan composition) precedes the decrease in glycated hemoglobin (HbA1c) associated with successful glycemic control, in some cases by as much as three weeks in mice. Thus, monitoring or measuring the change in the N-linked glycosylation pattern or composition of total blood or blood component proteins (or total N-glycan composition) in blood or blood component samples obtained from a diabetic individual or patient undergoing an anti-diabetic intervention therapy may be used as an early indicator of the decrease in HbA1c which is associated with successful glycemic control. Furthermore, detecting a change in N-linked glycosylation pattern or composition of blood or blood component proteins (or total N-glycan composition) during anti-diabetic intervention therapy may be used to evaluate the efficacy of the intervention therapy at a selected point in time, independent of determining the change in HbA1c, and thereby permit earlier adjustment of the frequency or dose of the intervention therapy to maintain glycemic control. In addition, detecting a difference in N-linked glycosylation pattern or composition of blood or blood component proteins (or total N-linked glycan composition) in a subject as compared to a normal (i.e., non-diabetic or normoglycemic) N-linked glycosylation pattern or composition of blood or blood component proteins (or total N-linked glycan composition) may be used to diagnose diabetes or pre-diabetes in the subject.

In one aspect, the present invention therefore provides a method of monitoring a level of glycemic control in a subject during anti-diabetic therapy or treatment comprising (a) providing an N-glycan composition of a blood or blood component sample obtained from the subject at a first time-point during the anti-diabetic therapy or treatment; and (b) determining an N-glycan composition of a blood or blood component sample obtained from the subject at a second time-point during the anti-diabetic therapy or treatment, wherein the second time-point is subsequent to the first time-point, wherein a difference between the N-glycan composition of the blood or blood component sample at the second time-point and the N-glycan composition of the blood or blood component sample at the first time-point indicates an increased or decreased level of glycemic control at the second time-point compared to the first time-point. In certain embodiments, the difference in N-glycan composition may be detected as a quantitative increase or decrease in the amount of one or more N-glycans or as a trend of increasing or decreasing amount of one or more N-glycans, regardless of the statistical significance of the difference. Alternatively, the difference in N-glycan composition may be detected as a statistically significant increase or decrease in the amount of one or more N-glycans.

In a further aspect, the method of monitoring the level of glycemic control in a subject during anti-diabetic therapy or treatment comprises (a) providing an N-glycan composition of a blood or blood component sample obtained from the subject at a first time-point during the anti-diabetic therapy or treatment; and (b) determining an N-glycan composition of a blood or blood component sample obtained from the subject at a second time-point during the anti-diabetic therapy or treatment, wherein the second time point is subsequent to the first time-point; wherein an increased level of glycemic control at the second time-point compared to the first time-point is indicated by
i) a decrease in an amount of at least one high mannose N-glycan, hybrid N-glycan, complex N-glycan, and/or O-acetylated N-glycan in the N-glycan composition of the blood or blood component sample at the first time-point as compared to an amount of a corresponding N-glycan in the N-glycan composition of the blood or blood component sample at the second time-point, and/or;
ii) an increase in an amount of one or more fucosylated N-glycans in the N-glycan composition of the blood or blood component sample at the first time-point as compared to an amount of a corresponding fucosylated N-glycan in the N-glycan composition of the blood or blood component sample at the second time-point.

In a particular embodiment of any of the above methods of monitoring the level of glycemic control in a subject during anti-diabetic therapy or treatment, the high mannose N-glycans are selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000). In particular embodiments of the above, the hybrid N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc. In another particular embodiment of the above, the complex N-glycan is Sia₂Gal₂GlcNAc₂Man₃GlcNAc₂ (540020), wherein Sia is Neu5Ac or Neu5Gc. In another particular embodiment of any of the above methods, the O-acetylated (O-Ac) N-glycans are selected from the group consisting of Sia₂Gal₂GlcNAc₂Man₃GlcNAc₂ (1 O-Ac) (540021), Sia₂Gal₂GlcNAc₂Man₃GlcNAc₂ (2 O-Ac) (540022), Sia₃Gal₂GlcNAc₂Man₃GlcNAc₂ (1 O-Ac) (540031), and Sia₃Gal₂GlcNAc₂Man₃GlcNAc₂ (2 O-Ac) (540032), wherein Sia is Neu5Ac or Neu5Gc. In another particular embodiment of any of the above methods, the fucosylated N-glycans are selected from the group consisting of Sia₃Gal₃GlcNAc₃Man₃GlcNAc₂ (Fuc) (651030), Sia₃Gal₃GlcNAc₃Man₃GlcNAc₂ (Fuc) (1 O-Ac) (651031), and Sia₄Gal₄GlcNAc₄Man₃GlcNAc₂ (Fuc) (761040), wherein Sia is Neu5Ac or Neu5Gc. It is to be understood that any of the foregoing specific N-glycans, or any combination thereof, may be evaluated in any of the foregoing methods for monitoring glycemic control.

In any of the foregoing methods of monitoring the level of glycemic control in a subject during anti-diabetic therapy or treatment, the difference in N-glycan composition may be detected as a quantitative increase or decrease in the amount of the one or more N-glycans or as a trend of increasing or decreasing amount of the one or more N-glycans, regardless of the statistical significance of the difference. Alternatively, the difference in N-glycan composition may be detected as a statistically significant increase or decrease in the amount of the one or more N-glycans.

In another aspect, the present invention provides a method of diagnosing diabetes mellitus or pre-diabetes in a subject comprising (a) determining an N-glycan composition of a blood or blood component sample obtained from the subject; and (b) comparing the N-glycan composition of the blood or blood component sample of the subject to an N-glycan composition of a normoglycemic blood or blood component, wherein a difference between the N-glycan composition of the blood or blood component sample from the subject and the N-glycan composition of the normoglycemic blood or blood component indicates diabetes mellitus or pre-diabetes in the subject.

In these diagnostic methods, the N-glycan composition of the normoglycemic blood or blood component for use in the comparison may be a normal N-glycan composition representative of the non-diabetic, non-pre-diabetic population in general, or it may be an N-glycan composition of a blood or blood component previously obtained from the subject (i.e., prior to development of diabetes or pre-diabetes). Accordingly, the present invention further provides a method of diagnosing diabetes mellitus or pre-diabetes in a subject comprising (a) determining an N-glycan composition of a blood or blood component sample obtained from the subject; and (b) comparing the N-glycan composition of the blood or blood component sample of the subject to an N-glycan composition of a normoglycemic blood or blood component sample previously obtained from the subject, wherein a difference between the N-glycan composition of the blood or blood component sample from the subject and the N-glycan composition of the normoglycemic blood or blood component sample indicates diabetes mellitus or pre-diabetes in the subject.

In a further aspect, the methods of diagnosing diabetes mellitus or pre-diabetes in a subject comprise (a) determining an N-glycan composition of a blood or blood component sample obtained from the subject; and (b) comparing the N-glycan composition of the blood or blood component sample of the subject to an N-glycan composition of a normoglycemic blood or blood component, wherein diabetes mellitus or pre-diabetes is indicated by
i) an increase in an amount of at least one high mannose N-glycan, hybrid N-glycan, complex N-glycan, and/or O-acetylated N-glycan in the N-glycan composition of the blood or blood component sample of the subject as compared to an amount of a corresponding N-glycan in the N-glycan composition of the normoglycemic blood or blood component sample, and/or;
ii) a decrease in an amount of one or more fucosylated N-glycans in the N-glycan composition of the blood or blood component sample of the subject as compared to an amount of a corresponding fucosylated N-glycan in the N-glycan composition of the normoglycemic blood or blood component.

In a particular embodiment of any of the above methods for diagnosing diabetes mellitus in a subject, the high mannose N-glycans are selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000). In particular embodiments of the above, the hybrid N-glycans selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc. In another particular embodiment of the above, the complex N-glycan is Sia₂Gal₂GlcNAc₂Man₃GlcNAc₂ (540020), wherein Sia is Neu5Ac or Neu5Gc. In another particular embodiment of any of the above methods, the O-acetylated (O-Ac) N-glycans are selected from the group consisting of Sia₂Gal₂GlcNAc₂Man₃GlcNAc₂ (1 O-Ac) (540021), Sia₂Gal₂GlcNAc₂Man₃GlcNAc₂ (2 O-Ac) (540022), Sia₃Gal₂GlcNAc₂Man₃GlcNAc₂ (1 O-Ac) (540031), and Sia₃Gal₂GlcNAc₂Man₃GlcNAc₂ (2 O-Ac) (540032), wherein Sia is Neu5Ac or Neu5Gc. In another particular embodiment of any of the above methods, the fucosylated N-glycans are selected from the group consisting of Sia₃Gal₃GlcNAc₃Man₃GlcNAc₂ (Fuc) (651030), Sia₃Gal₃GlcNAc₃Man₃GlcNAc₂ (Fuc) (1 O-Ac) (651031), and Sia₄Gal₄GlcNAc₄Man₃GlcNAc₂ (Fuc) (761040), wherein Sia is Neu5Ac or Neu5Gc. It is to be understood that any of the foregoing specific N-glycans, or any combination thereof, may be evaluated in any of the foregoing methods for diagnosing diabetes mellitus or pre-diabetes.

In any of the foregoing methods for diagnosing diabetes mellitus or pre-diabetes in a subject, the difference in N-glycan composition may be detected as a quantitative increase or decrease in the amount of the one or more N-glycans or as a trend of increasing or decreasing amount of the one or more N-glycans, regardless of the statistical significance of the difference. Alternatively, the difference in N-glycan composition may be detected as a statistically significant increase or decrease in the amount of the one or more N-glycans.

In further embodiments of any of the above methods, the N-glycan composition is determined by separating the N-glycans from the proteins in blood or a blood component to provide a composition of N-glycans, and determining the relative amounts of N-glycans in the composition. In one aspect the determination of relative amounts of N-glycans in the composition is accomplished using Matrix Adsorption Laser Desorption/Ionization-Time-Of-Flight mass spectrometry (MALDI-TOF MS). In a further embodiment, the MALDI-TOF MS provides data that is analyzed by a computer to provide the N-glycan composition. Alternatively, the determination of relative amounts of N-glycans in the composition is accomplished using any quantitative or semi-quantitative analytical method for analysis of N-glycans, such as HPLC, capillary electrophoresis or immunoassay. The quantitative or semi-quantitative data provided by the analytical method may be analyzed by a computer to provide the N-glycan composition.

In addition, the present invention provides N-glycan biomarkers for monitoring a level of glycemic control in a subject during anti-diabetic therapy or treatment. Also provided is the use of at least one N-glycan biomarker for monitoring a level of glycemic control in a subject during anti-diabetic therapy or treatment. Further provided is the use of an amount of at least one N-glycan biomarker in the blood or blood component of a subject during anti-diabetic therapy or treatment as an indicator of a level of glycemic control in the subject. The present invention further provides for the use of an amount of one or more high mannose N-glycans, hybrid N-glycans, O-acetylated N-glycans, complex N-glycans, fucosylated N-glycans, or combinations thereof, in a blood or blood component sample obtained from a subject during anti-diabetic therapy or treatment for monitoring the level of glycemic control in the subject.

Further, the present invention provides N-glycan biomarkers for diagnosing diabetes mellitus or pre-diabetes in a subject. Also provided is the use of at least one N-glycan biomarker for diagnosing diabetes mellitus or pre-diabetes in a subject. Further provided is the use of an amount of at least one N-linked glycan biomarker in the blood or blood component of a subject for diagnosis of diabetes mellitus or pre-diabetes in the subject. The present invention further provides for the use of an amount of one or more high mannose N-glycans, hybrid N-glycans, O-acetylated N-glycans, complex N-glycans, fucosylated N-glycans, or combinations thereof, in a blood or blood component sample obtained from a subject for diagnosis of diabetes mellitus or pre-diabetes in the subject.

In a further aspect, the present invention provides kits for determining an N-glycan composition of a blood or blood component sample, wherein the N-glycan composition comprises one or more of a high mannose N-glycan, a hybrid N-glycans, an O-acetylated N-glycan, a complex N-glycan, a fucosylated N-glycan, or combinations thereof. The N-glycan composition thus determined may be used to monitor glycemic control or to diagnose diabetes or pre-diabetes.

### BRIEF DESCRIPTION OF THE DRAWINGS

FIG. 1 shows the nomenclature developed by the Consortium of Functional Glycomics for representing glycan structures.

FIGS. 2A-2E show that various high mannose N-glycans were lower in rosiglitazone-treated db/db mice compared to vehicle-treated db/db mice. The graphs plot the median of all samples over time, with error bars representing the 25/75 percentile range. Statistical significant of the difference between rosiglitazone-treated and vehicle-treated db/db mice at each time point is indicated by asterisks, where *=p<0.005, **=p<0.01 and ***=p<0.001. FIG. 2A illustrates the rosiglitazone response of glycan 520000. FIG. 2B illustrates the rosiglitazone response of glycan 620000. FIG. 2C illustrates the rosiglitazone response of glycan 720000. FIG. 2D illustrates the rosiglitazone response of glycan 820000. FIG. 2E illustrates the rosiglitazone response of glycan 920000.

FIGS. 3A-3C show that various fucosylated N-glycans were higher in rosiglitazone-treated db/db mice compared to vehicle-treated db/db mice. The graphs plot the median of all samples over time, with error bars representing the 25/75 percentile range. Statistical significance of the difference between rosiglitazone-treated and vehicle-treated db/db mice at each time point is indicated by asterisks, where *=p<0.05, **=p<0.01, and ***=p<0.001. FIG. 3A illustrates the rosiglitazone response of glycan 651030. FIG. 3B illustrates the rosiglitazone response of glycan 651031. FIG. 3C illustrates the rosiglitazone response of glycan 761040. Glycan 761040 was below the limit of quantitation (LOQ) in some samples, preventing statistical analysis at some time points. Approximate statistical significance of the difference between rosiglitazone-treated and vehicle-treated db/db mice at each time point is indicated by asterisks, where *=p<0.05, **=p<0.01, and ***=p<0.001.

FIGS. 4A-4D show that various O-acetylated N-glycans were lower in rosiglitazone-treated db/db mice compared to vehicle-treated db/db mice. The graphs plot the median of all samples over time, with error bars representing the 25/75 percentile range. Statistical significance of the difference between rosiglitazone-treated and vehicle-treated db/db mice at each time point is indicated by asterisks, where *=p<0.05, **=p<0.01, and ***=p<0.001. FIG. 4A illustrates the rosiglitazone response of glycan 540021. FIG. 4B illustrates the rosiglitazone response of glycan 540022. FIG. 4C illustrates the rosiglitazone response of glycan 540031. FIG. 4D illustrates the rosiglitazone response of glycan 540032.

FIGS. 5A-5C show that various hybrid N-glycans were lower in rosiglitazone-treated db/db mice compared to vehicle-treated db/db mice. The graphs plot the median of all samples over time, with error bars representing the 25/75 percentile range. Statistical significance of the difference between rosiglitazone-treated and vehicle-treated db/db mice at each time point is indicated by asterisks, where *=p<0.05, **=p<0.01, and ***=p<0.001. FIG. 5A illustrates the rosiglitazone response of glycan 430010. FIG. 5B illustrates the rosiglitazone response of glycan 530010. FIG. 5C illustrates the rosiglitazone response of glycan 630010.

FIG. 6 shows that glycan 540020 is lower in rosiglitazone-treated db/db mice compared to vehicle-treated db/db mice. The graph plots the median of all samples over time, with error bars representing the 25/75 percentile range. Statistical significance of the difference between rosiglitazone-treated and vehicle-treated db/db mice at each time point is indicated by asterisks, where *=p<0.05, **=p<0.01, and ***=p<0.001.

FIGS. 7A-7E are scatter plots showing that various high mannose N-glycans were lower in rosiglitazone-treated db/db mice compared to vehicle-treated db/db mice in Study 2, which confirms the results of Study 1. Statistical significance of the difference between rosiglitazone-treated and vehicle-treated db/db mice at Day 7 is indicated by asterisks, where *=p<0.05, **=p<0.01, and ***=p<0.001. FIG. 7A shows that Glycan 520000 is lower in rosiglitazone-treated db/db mice compared to vehicle-treated db/db mice. FIG. 7B shows that Glycan 620000 is lower in rosiglitazone-treated db/db mice compared to vehicle-treated db/db mice. FIG. 7C shows that Glycan 720000 is lower in rosiglitazone-treated db/db mice compared to vehicle-treated db/db mice. FIG. 7D shows that Glycan 820000 is lower in rosiglitazone-treated db/db mice compared to vehicle-treated db/db mice. FIG. 7E shows that Glycan 920000 is lower in rosiglitazone-treated db/db mice compared to vehicle-treated db/db mice.

FIGS. 8A-8C are scatter plots showing that various fucosylated N-glycans were higher in rosiglitazone-treated db/db mice compared to vehicle-treated db/db mice in Study 2, which confirms the results of Study 1. Statistical significance of the difference between rosiglitazone-treated and vehicle-treated db/db mice at Day 7 is indicated by asterisks, where *=p<0.05, **=p<0.01, and ***=p<0.001. FIG. 8A show that Glycan 651030 exhibits a significant increase in rosiglitazone-treated db/db mice compared to vehicle-treated db/db mice. FIG. 8B shows that Glycan 651031 exhibits a significant increase in rosiglitazone-treated db/db mice compared to vehicle-treated db/db mice. FIG. 8C shows that Glycan 761040 exhibits a significant increase in rosiglitazone-treated db/db mice compared to vehicle-treated db/db mice.

FIGS. 9A-9D are scatter plots showing that various O-acetylated N-glycans were lower in rosiglitazone-treated db/db mice compared to vehicle-treated db/db mice in Study 2, which confirms the results of Study 1. Statistical significance of the difference between rosiglitazone-treated and vehicle-treated db/db mice at Day 7 is indicated by asterisks, where *=p<0.05, **=p<0.01, and ***=p<0.001. FIG. 9A shows that Glycan 540021 is lower in rosiglitazone-treated db/db mice compared to vehicle-treated db/db mice. FIG. 9B shows that Glycan 540022 is lower in rosiglitazone-treated db/db mice compared to vehicle-treated db/db mice. FIG. 9C shows that Glycan 540031 is lower in rosiglitazone-treated db/db mice compared to vehicle-treated db/db mice. FIG. 9D shows that Glycan 540032 is lower in rosiglitazone-treated db/db mice compared to vehicle-treated db/db mice.

FIGS. 10A-10C are scatter plots showing that various hybrid N-glycans were lower in rosiglitazone-treated db/db mice compared to vehicle-treated db/db mice in Study 2, which confirms the results of Study 1. Statistical significance of the difference between rosiglitazone-treated and vehicle-treated db/db mice at Day 7 is indicated by asterisks, where *=p<0.05, **=p<0.01, and ***=p<0.001. FIG. 10A shows that Glycan 430010 is lower in rosiglitazone-treated db/db mice compared to vehicle-treated db/db mice. FIG. 10B shows that Glycan 530010 is lower in rosiglitazone-treated db/db mice compared to vehicle-treated db/db mice. FIG. 10C shows that Glycan 630010 is lower in rosiglitazone-treated db/db mice compared to vehicle-treated db/db mice.

FIG. 11 is a scatter plot showing that Glycan 540020 is lower in rosiglitazone-treated db/db mice compared to vehicle-treated db/db mice in Study 2, which confirms the results of Study 1. Statistical significance of the difference between rosiglitazone-treated and vehicle-treated db/db mice at Day 7 is indicated by asterisks, where *=p<0.05, **=p<0.01, and ***=<0.001.

FIGS. 12A-12D show that various high mannose N-glycans were lower in insulin detemir-treated db/db mice compared to vehicle-treated db/db mice. The graphs plot the mean of all samples over time, with error bars representing the standard error. Statistical significance of the difference between insulin detemir-treated and vehicle-treated db/db mice at each time point is indicated by asterisks, where *=p<0.05, **=p<0.01, and ***=p<0.001. FIG. 12A shows that Glycan 520000 is lower in insulin detemir-treated db/db mice compared to vehicle-treated db/db mice. FIG. 12B shows that Glycan 620000 is lower in insulin detemir-treated db/db mice compared to vehicle-treated db/db mice. FIG. 12C shows that Glycan 720000 is lower in insulin detemir-treated db/db mice compared to vehicle-treated db/db mice. FIG. 12D shows that Glycan 820000 is lower in insulin detemir-treated db/db mice compared to vehicle-treated db/db mice.

FIGS. 13A-13C show that various hybrid N-glycans were lower in insulin detemir-treated db/db mice compared to vehicle-treated db/db mice. The graphs plot the mean of all samples over time, with error bars representing the standard error. Statistical significance of the difference between insulin detemir-treated and vehicle-treated db/db mice at each time point is indicated by asterisks, where *=p<0.05, **=p<0.01, and ***=p<0.001. FIG. 13A shows that Glycan 430010 is lower in insulin detemir-treated db/db mice compared to vehicle-treated db/db mice. FIG. 13B shows that Glycan 530010 is lower in insulin detemir-treated db/db mice compared to vehicle-treated db/db mice. FIG. 13C shows that Glycan 630010 is lower in insulin detemir-treated db/db mice compared to vehicle-treated db/db mice.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

As used herein, the terms “N-glycan” and “N-linked glycan” are used interchangeably and refer to an N-glycan in which the N-acetylglucosamine residue at the reducing end that may be linked in a β1 linkage to the amide nitrogen of an asparagine residue of an attachment group in the protein. Thus, the term refers to the N-glycan whether it is attached to the protein or has been detached from the protein. N-glycans are oligosaccharides that have a common pentasaccharide core of Man₃GlcNAc₂ (“Man” refers to mannose; “Glc” refers to glucose; and “NAc” refers to N-acetyl; GlcNAc refers to N-acetylglucosamine). Usually, N-glycan structures are presented with the non-reducing end to the left and the reducing end to the right. The reducing end of the N-glycan is the end that may be attached to the Asn residue comprising the glycosylation site on the protein. N-glycans differ with respect to the number of branches (antennae) comprising peripheral sugars (e.g., GlcNAc, galactose, fucose and sialic acid) that are added to the Man₃GlcNAc₂ (“Man₃”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid (“Sia”) or derivatives (e.g., “NANA” or “NeuAc” where “Neu” refers to neuraminic acid and “Ac” refers to acetyl, or the derivative NGNA, which refers to N-glycolylneuraminic acid). Complex N-glycans may also have intrachain substitutions comprising “bisecting” GlcNAc and core fucose (“Fuc”). Complex N-glycans may also have multiple antennae on the “trimannose core,” often referred to as “multiple antennary N-glycans.” A “hybrid” N-glycan has at least one GlcNAc on the terminal of the 1,3 mannose arm of the trimannose core, no GlcNAc on the 1,6 mannose arm, and zero or more mannoses on the 1,6 mannose arm of the trimannose core. N-glycans consisting of a Man₃GlcNAc₂ structure are called paucimannose. The term “fucosylated glycan” or “fucosylated N-glycan” refers to any N-glycan that has one or more fucose residue(s) anywhere on the structure, including, but not limited to core fucose. The term “O-acetylated glycan” or “O-acetylated N-glycan” refers to any N-glycan that has one of the hydroxyl groups esterified with an acetyl group or more than one hydroxyl group, each esterified with an acetyl group. The various N-glycans are also referred to as “glycoforms.”

As used herein, the terms “N-linked glycosylated” and “N-glycosylated” are used interchangeably and refer to an N-glycan attached to an attachment group comprising an asparagine residue or an N-linked glycosylation site or motif.

As used herein, the term “N-linked glycosylation profile” refers to the N-linked glycosylation pattern or signature of blood or a blood component and comprises a quantitation of the relative amounts of the N-glycans detected in a blood or blood component sample.

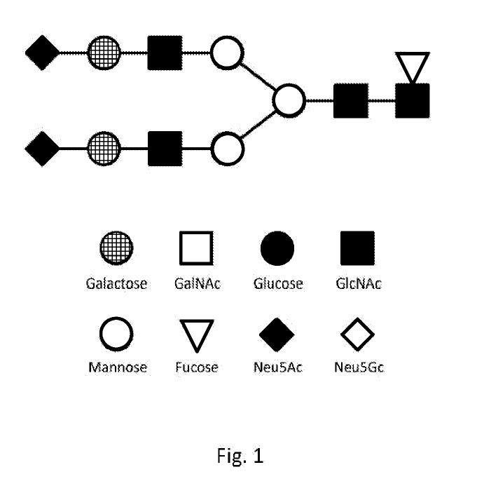

As used herein, the structure of an N-glycan may be expressed using a six-digit identifier. The six-digit identifiers are interpreted as follows: the first digit indicates the number of hexoses in the structure (i.e., mannose, galactose or glucose); the second digit indicates the number of N-acetylhexosamines in the structure (i.e., GlcNAc or GalNAc); the third digit indicates the number of deoxyhexoses in the structure (i.e., fucose); the fourth digit indicates the number of N-acetylneuraminic acids (Neu5Ac) in the structure; the fifth digit indicates the number of N-glycolylneuraminic acids (Neu5Gc) in the structure, and; the sixth digit indicates the number of O-acetates (OAc) in the structure. Alternatively, the structure of an N-glycan may be illustrated using the nomenclature developed by the Consortium of Functional Glycomics, as is known in the art and illustrated in FIG. 1.

As used herein, the term “corresponding N-glycan” and its equivalents refers to detected amounts of a particular N-glycan under a first set of conditions as compared to detected amounts of the same N-glycan under a second set of conditions. For example, comparison of amounts of high mannose glycan Man₉GlcNAc₂ (920000) in blood or a blood component of a diabetic subject to amounts of high mannose glycan Man₉GlcNAc₂ (920000) in blood or a blood component of a normoglycemic subject is a comparison to the corresponding N-glycan.

As used herein, the term “blood” refers to whole blood. The term “blood component” refers to an acellular liquid fraction of whole blood, and includes both serum and plasma. The terms “blood component,” “serum” and “plasma” and their equivalents are used interchangeably herein. As the proteins carrying the N-glycans of interest in the inventive methods can be found in any of whole blood, serum or plasma, any of these sample types can be used as a source of N-glycans for analysis.

As used herein, the term “pre-diabetes” refers to a condition in which blood glucose levels ace higher than normal but not yet high enough to be diagnosed as diabetes. The term “hyperglycemia” also refers to blood glucose levels that are higher than normal, and includes pre-diabetes.

As used herein, the term “normoglycemic” refers to the normal blood glucose level in humans. This range is typically between about 3.6 and 5.8 mM, or 64.8 and 104.4 mg/dL. Values outside these ranges may be an indicator of a medical condition, such as diabetes, pre-diabetes, hyperglycemia or hypoglycemia.

The present invention provides biomarkers for determining the level of glycemic control in a subject during anti-diabetic therapy or treatment. In addition, the invention provides biomarkers for diagnosing diabetes or pre-diabetes in a subject. The biomarkers comprise the N-linked glycosylation profile of total blood or blood component proteins in a blood or blood component sample obtained from a subject at a time-point during anti-diabetic therapy or treatment, wherein an amount of one or more particular N-glycans in the profile increases or decreases as compared to the N-linked glycosylation profile of total blood or blood component proteins in a blood or blood component sample obtained from the subject at a prior time-point during anti-diabetic therapy or treatment regime. In addition, the biomarkers comprise the N-linked glycosylation profile of total blood or blood component proteins in a blood or blood component sample obtained from a subject, wherein an amount of one or more particular N-glycans in the profile is increased or decreased as compared to an N-linked glycosylation profile of total blood or blood component proteins in a normoglycemic blood or blood component sample.

Results in the db/db mouse model suggest that, in general, the increase and/or decrease in the amounts of particular N-glycans in the serum may occur between 3 and 14 days after the start of the anti-diabetic therapy or treatment. Similarly, an increase and/or decrease in the amounts of particular N-glycans in the serum in response to a change in frequency or dose of the anti-diabetic therapy or treatment, in response to a change in environment which alters the level of glycemic control, or in response to development of resistance to the anti-diabetic drug may also occur between 3 and 14 days after such change. Thus, the present invention provides a biomarker for evaluating the level of glycemic control of an anti-diabetic therapy or treatment regime over time during anti-diabetic therapy or treatment.

While glycemic control is routinely evaluated by monitoring changes in HbA1c levels over time in patients undergoing anti-diabetic therapy or treatment, in general the changes in HbA1c levels are delayed relative to changes in the frequency or dose of the anti-diabetic therapy or treatment, changes in environment, or development of drug resistance. Therefore, it is not possible until some time after the event that changes the level of glycemic control to know that anti-diabetic therapy or treatment should be modified. To improve therapy or treatment outcomes, it would be desirable to know at an earlier time-point whether the particular therapy or treatment continues to be efficacious for glycemic control, allowing a non-efficacious therapy or treatment to be modified or replaced with another therapy or treatment at an earlier time period than is currently possible. As disclosed herein, the inventors have discovered that the N-linked glycan pattern, profile, or signature of total blood or blood component proteins may be used as a biomarker of changes in HbA1c amounts in blood or blood components at an earlier time-point. Thus, the present invention provides a biomarker that enables the level of glycemic control afforded by an anti-diabetic therapy or treatment regime to be determined at a time-point preceding the change in HbA1c amounts in blood or blood components.

These results demonstrate that the change in the N-linked glycosylation pattern or N-glycan profile of total blood or blood component proteins over time in an individual or patient undergoing an anti-diabetic therapy or treatment can be used as a biomarker for evaluating the efficacy of the anti-diabetic therapy or treatment. The observed correlation of the N-glycan changes with reduction in HbA1c levels, in particular, indicates that these biomarkers can be used to effectively monitor levels of glycemic control in diabetic subjects during anti-diabetic therapy or treatment. For monitoring glycemic control, a blood or blood component test sample is obtained from a subject undergoing an anti-diabetic therapy or treatment. The sample is treated to release the N-glycans from the proteins, for example with an enzyme such as PNGase-F. The N-glycans are then separated from the proteins to provide a composition of the N-glycans, which is then analyzed to determine the N-glycan pattern or profile for the blood or blood component sample. In one embodiment, the blood or blood component sample may be analyzed by MALDI-TOF MS, and the MALDI-TOF MS data may be analyzed by computer using a bioinformatics analysis program and the results of the analysis provided in a report showing the N-glycan pattern or profile for the blood or blood component sample. In an alternative embodiment, the sample may be analyzed by any means which provides the N-glycan pattern or profile of the sample, for example, HPLC, capillary electrophoresis or immunoassay. The N-glycan pattern or profile of the blood or blood component test sample is compared to the N-glycan pattern or profile of a blood or blood component reference sample obtained from the subject at a time-point in the anti-diabetic therapy or treatment prior to the test sample (i.e., the test sample is from a time-point in therapy that is subsequent to the reference sample). A change in the N-glycan pattern or profile of the test sample as compared to the N-glycan pattern or profile of the reference sample indicates a change in the level of glycemic control between the two time-points.

Immunoassay methods for monitoring levels of glycemic control in diabetic subjects during anti-diabetic therapy or treatment include any antibody-based assays for detection of the N-glycan of interest (i.e., the antibody target), for example enzyme-linked immunosorbent assays (ELISAs). Immunoassays employ polyclonal or monoclonal antibodies which specifically bind the target to detect the target by means of a detectable label. Detection may be either qualitative (presence or absence of the target) or quantitative (amount of the target). For the immunoassays of the invention the antibody will be specific for binding to an N-glycan associated with an increase or decrease in glycemic control as discussed above. The antibody may specifically recognize and bind to an epitope of the N-glycan, or it may specifically recognize and bind to an epitope comprising the N-glycan and the peptide or protein to which it is linked (i.e., a glycopeptide or glycoprotein epitope). The immunoassays for monitoring levels of glycemic control may also be in the form of a panel of immunoassays in which multiple antibodies targeting multiple N-glycans associated with levels of glycemic control (or the glycopeptides/glycoproteins to which they are linked) are used for detection and monitoring the level of glycemic control in a diabetic subject during anti-diabetic therapy or treatment.

It is to be understood that an increase or decrease in the amount of an N-glycan between two time-points during anti-diabetic therapy or treatment as disclosed herein may indicate either improved or reduced glycemic control. For example, if the N-glycans in the prior sample are present in amounts indicative of normal glucose levels, and at least one high mannose N-glycan, hybrid N-glycan, complex N-glycan, and/or O-acetylated N-glycan in the first sample is increased relative to the prior sample, and/or at least one fucosylated N-glycan in the first sample is decreased relative to the prior sample, reduced glycemic control between the two time-points is indicated. Conversely, if the N-glycans in the prior sample are present in amounts indicative of hyperglycemia, and at least one high mannose N-glycan, hybrid N-glycan, complex N-glycan, and/or O-acetylated N-glycan in the first sample is decreased relative to the prior sample, and/or at least one fucosylated N-glycan in the first sample is increased relative to the prior sample, improved glycemic control between the two time-points is indicated. Similarly, if the N-glycans in the prior sample are present in amounts indicative of hypoglycemia, and at least one high mannose N-glycan, hybrid N-glycan, complex N-glycan, and/or O-acetylated N-glycan in the first sample is increased relative to the prior sample, but does not substantially exceed amounts indicative of normal glucose levels, and/or at least one fucosylated N-glycan in the first sample is decreased relative to the prior sample, but does not fall substantially below amounts indicative of normal glucose levels, improved glycemic control between the two time-points may be indicated.

In certain aspects, the increase or decrease in amounts of N-glycans indicative of the level of glycemic control comprises an increase or decrease in one or more N-glycan selected from the group consisting of high mannose N-glycans including Man₉GlcNAc₂ (920000), Man₈GlcNAc₂ (820000), Man₇GlcNAc₂ (720000), Man₆GlcNAc₂ (620000), and Man₅GlcNAc₂ (520000); hybrid N-glycans including SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc; O-acetylated (O-Ac) N-glycans including Sia₂Gal₂GlcNAc₂Man₃GlcNAc₂ (1 O-Ac) (540021), Sia₂Gal₂GlcNAc₂Man₃GlcNAc₂ (2 O-Ac) (540022), Sia₃Gal₂GlcNAc₂Man₃GlcNAc₂ (1 O-Ac) (540031), and Sia₃Gal₂GlcNAc₂Man₃GlcNAc₂ (2 O-Ac) (540032), wherein Sia is Neu5Ac or Neu5Gc; complex N-glycans including Sia₂Gal₂GlcNAc₂Man₃GlcNAc₂ (540020), wherein Sia is Neu5Ac or Neu5Gc; and fucosylated N-glycans including Sia₃Gal₃GlcNAc₃Man₃GlcNAc₂ (Fuc) (651030), Sia₃Gal₃GlcNAc₃Man₃GlcNAc₂ (Fuc) (1 O-Ac) (651031), and Sia₄Gal₄GlcNAc₄Man₃GlcNAc₂ (Fuc) (761040), wherein Sia is Neu5Ac or Neu5Gc. Improved glycemic control as represented by a reduction in hyperglycemia is typically indicated by a decrease in one or more of the high mannose, hybrid, O-acetylated and/or complex N-glycans identified above, and/or by an increase in one or more of the fucosylated N-glycans identified above.

As the goal of glycemic control is to maintain blood glucose levels in a subject on anti-diabetic therapy or treatment at or near normal (i.e., normoglycemic) blood glucose levels, in certain embodiments the present methods for monitoring glycemic control provide a means for maintaining and adjusting the frequency and/or dose of anti-diabetic therapy which as closely as possible approximates amounts of N-glycans and an N-glycan profile representative of normoglycemic blood or blood components. Accordingly, when the analysis of N-glycans at a given time-point during anti-diabetic therapy or treatment reveals amounts of N-glycans and/or an N-glycan profile that correspond closely to normal HbA1c levels, glycemic control is achieved. The amounts of N-glycans and/or the N-glycan profile will therefore correspond to about 2-7 percent HbA1c or about 3-6 percent HbA1c. Amounts of N-glycans and/or an N-glycan profile that correspond to less than 2 percent or less than 3 percent HbA1c indicate a lack of glycemic control in the direction of hypoglycemia, and amounts of N-glycans and/or an N-glycan profile that correspond to greater than 6 percent or greater than 7 percent HbA1c indicate a lack of glycemic control in the direction of hyperglycemia.

In further embodiments, the two blood or blood component samples for monitoring of the level of glycemic control are obtained from the subject at time-points during anti-diabetic therapy or treatment selected from 3, 7, 14, 21, 28, 35, 42, 49 or 56 days apart. However, it should be understood that the N-glycan profile at any time-point during anti-diabetic therapy or treatment may be compared to any prior time-point to monitor the level of glycemic control.

In particular embodiments of the above, the anti-diabetic therapy or treatment comprises an insulin, an insulin sensitizer, insulin secretagogue, alpha-glucosidase inhibitor, incretin or incretin mimetic, dipeptidyl peptidase 4 (DPP4) inhibitor, amylin or amylin analog, or GLP-1 receptor agonist. Insulin sensitizers include but are not limited to biguanides and thiazolidinediones wherein the biguanides include but are not limited to metformin, phenformin, and buformin and the thiazolidinediones include but are not limited to rosiglitazone, pioglitazone, and troglitazone. The insulin secretagogues include but are not limited to sulfonylureas and non-sulfonylureas wherein the sulfonylureas include but are not limited to tolbutamide, acetohexamide, tolazamide, chlorpropamide, glipizide, glyburide, glimepiride, and gliclazide and the non-sulfonylurease include but are not limited to metglitinides such as repaglinide and nateglinide. Alpha-glucosidase inhibitors include but are not limited to miglitol and acarbose. Incretin or incretin mimetics include but are not limited to GLP1 receptor agonists such as GLP1, oxyntomodulin, exenatide, liraglutide, taspoglutide, and glucagon analogs that have GLP1 receptor agonist activity. DPP4 inhibitors include but are not limited to vildagliptin, sitagliptin, saxagliptin, and linagliptin.

In addition, results of the present studies suggest that the increase and/or decrease in the amounts of particular N-glycans in blood or blood components may be used as an indicator of diabetes mellitus or pre-diabetes in a subject, i.e., as a diagnostic biomarker for diabetes mellitus or pre-diabetes. In this embodiment, an increase and/or decrease in the amounts of particular N-glycans in the blood or blood components of a subject, when compared to amounts of the corresponding N-glycan in normoglycemic blood or blood components, provides a diagnostic tool for diabetes or pre-diabetes. Specifically, N-glycans that are increased or decreased in a subject in comparison with levels of the corresponding N-glycans in normoglycemic subjects, or in comparison with levels of the corresponding N-glycans in the subject prior to developing diabetes or pre-diabetes, indicate a diagnosis of diabetes or pre-diabetes depending on the amount of such increase or decrease. Thus, the present invention provides biomarkers for diagnosing diabetes mellitus or pre-diabetes in a subject.

In a specific embodiment, the relative amounts of N-glycans in the N-linked glycosylation pattern or profile of total blood or blood component proteins have been found to be a useful diagnostic biomarker for diagnosing diabetes mellitus or pre-diabetes. The observed correlation of the N-glycan changes with increased or reduced HbA1c levels, in particular, indicates that these biomarkers can be used to effectively detect hyperglycemia, which is a leading indicator of diabetes and pre-diabetes.

In one example of the methods for diagnosing diabetes or pre-diabetes, a blood or blood component sample is obtained from a subject and is treated to release the N-glycans from the proteins, for example with an enzyme such as PNGase-F. The N-glycans are then separated from the proteins to provide a composition of the N-glycans, which is then analyzed to determine the N-glycan pattern or profile for the blood or blood component sample. In one embodiment, the sample may be analyzed by Matrix-Assisted Laser Desorption/Ionization-Time-Of-Flight mass spectrometry (MALDI-TOF MS), and the MALDI-TOF MS data may be analyzed by computer using a bioinformatics analysis program and the results of the analysis provided in a report showing the N-glycan pattern or profile for the sample. The blood or blood component sample of the subject may also be analyzed by any other means which provides the N-glycan pattern or profile of the sample, for example, HPLC, capillary electrophoresis or immunoassay.

Immunoassay methods for diagnosing diabetes or pre-diabetes include any antibody-based assays for detection of the N-glycan of interest (i.e., the antibody target), for example enzyme-linked immunosorbent assays (ELISAs). Immunoassays employ polyclonal or monoclonal antibodies which specifically bind the target to detect the target by means of a detectable label. Detection may be either qualitative (presence or absence of the target) or quantitative (amount of the target). For the immunoassays of the invention the antibody will be specific for binding to an N-glycan associated with an increase or decrease in glycemic control as discussed above. The antibody may specifically recognize and bind to an epitope of the N-glycan, or it may specifically recognize and bind to an epitope comprising the N-glycan and the peptide or protein to which it is linked (i.e., a glycopeptide or glycoprotein epitope). The immunoassays for diagnosing diabetes or pre-diabetes may also be in the form of a panel of immunoassays in which multiple antibodies targeting multiple N-glycans associated with a diagnosis of diabetes or pre-diabetes (or the glycopeptides/glycoproteins to which they are linked) are used for determining whether a subject is hyperglycemic, hypoglycemic or normoglycemic.

The N-glycan pattern or profile of the subject's blood or blood component sample is then compared to the N-glycan pattern or profile of normoglycemic blood or blood components. A change in the N-glycan pattern or profile of the subject sample as compared to the N-glycan pattern or profile of, normoglycemic blood or blood components indicates a diagnosis of diabetes mellitus or pre-diabetes depending on the extent of the change. Alternatively, the N-glycan pattern or profile of the subject's blood or blood component sample may be compared to the N-glycan pattern or profile of normoglycemic blood or blood components previously obtained from the subject. A change in the N-glycan pattern or profile of the subject sample as compared to the N-glycan pattern or profile of the normoglycemic blood or blood components previously obtained from the subject indicates a diagnosis of diabetes mellitus or pre-diabetes depending on the extent of the change.

Specifically, if the N-glycans in the blood or blood component sample of the subject are present in amounts comparable to the amounts of the corresponding N-glycans in normoglycemic blood components, normal glucose levels are indicated and a diagnosis of diabetes or pre-diabetes is not made. Conversely, if the N-glycans in the blood or blood component sample of the subject are present in amounts that are different from the amounts of the corresponding N-glycans in normoglycemic blood components, and are indicative of hyperglycemia, then a diagnosis of diabetes or pre-diabetes is made based on the extent of the difference. For example, if at least one high mannose N-glycan, hybrid N-glycan, complex N-glycan, and/or O-acetylated N-glycan in the subject sample is increased relative to amounts of the corresponding N-glycan in normoglycemic blood or blood components, and/or at least one fucosylated N-glycan in the subject sample is decreased relative to amounts of the corresponding N-glycan in normoglycemic blood or blood components, a diagnosis of diabetes or pre-diabetes is indicated. In a further example, if the amounts of high mannose N-glycan, hybrid N-glycan, complex N-glycan, O-acetylated N-glycan and/or fucosylated N-glycan in the subject sample are comparable to the amounts of the corresponding N-glycans in normoglycemic blood components, then a diagnosis of diabetes or pre-diabetes is not indicated.

As the diagnosis of diabetes or pre-diabetes is made in relation to normal blood glucose levels representative of a normoglycemic subject, in certain embodiments of the present methods for diagnosing diabetes the amounts of N-glycans and/or the N-glycan profile of the subject will be compared to amounts of N-glycans and/or an N-glycan profile that corresponds to less than 5.7 percent HbA1c, which corresponds to normal HbA1c levels. Amounts of at least one N-glycan and/or an N-glycan profile that corresponds to greater than or equal to 5.7 percent but less than 6.5 percent HbA1c indicate hyperglycemia or pre-diabetes. Amounts of at least one N-glycan and/or an N-glycan profile that corresponds to greater than or equal to 6.5 percent HbA1c indicate a diagnosis of diabetes.

In a specific embodiment of the methods for monitoring levels of glycemic control and diagnosing diabetes or pre-diabetes, sample preparation and analysis is as follows. Starting from complex biological samples (e.g., blood, plasma or serum), each sample is enzymatically treated to provide a crude mixture of released N-glycans, peptides, lipids, and nucleic acids. For example, the samples may be denatured and then digested with trypsin, followed by heat-inactivation, and then digestion with PNGase F (See for example, Papac, et al. Glycobiology 8: 445-454 (1998)). The N-glycans are captured to a solid support that is capable of binding N-glycans and does not bind proteins, polypeptides, peptides, lipids, nucleic acids, or other macromolecules present in the sample. In particular embodiments, the solid support are beads (as shown in the figure) comprising aminoxy-functionalized polymers (For example, BLOTGLYCO H beads, Sumitomo Bakelite Co., Ltd., Tokyo, Japan) and the N-glycans are bound thereto via oxime bond formation. After thorough washing to remove nonspecifically bound substances, the covalently bound N-glycans are subjected to on-bead methyl esterification to stabilize sialic acids (See for example, Sekiya et al., Anal. Chem. 77: 4962-4968 (2005)) and are recovered in the form of oxime derivatives of the O-substituted aminooxy compound that had been added. The N-glycans are simultaneously released from the substrate, labeled and analyzed by MALDI-TOF MS in the positive-ion, reflector mode. Methods for performing MALDI-TOF MS analysis of N-glycans have been disclosed for example in Miele et al. Biotechnol. Appl. Biochem. 25: 151-157 (1997). The results may be analyzed by computer using a bioinformatics program. For example, the detected N-glycan peaks in MALDI-TOF-MS spectra may be picked by means of a computer using a software such as FlexAnalysis version 3 (Bruker Daltonics, Billerica, Mass.). Glycan structures may be identified using GlycoMod Tool and GlycoSuite (Tyrian Diagnostics Limited, Sydney, Australia). The above process has been disclosed in the art, for example Nishimura et al. (Angew Chem. Int. Ed. Engl., 44: 91-96 (2004)); Niikura et al. (Chem.-A Eur. J. 11: 3825-3834 (2005); Furukawa et al. (Anal. Chem., 80: 1094-1101 (2008)); Miura et al. (Chem.-A Eur. J, 13: 4797-4804 (2007)); Shimaoka et al. (Chem.-A Eur. J. 13: 1664-1673 (2007)); Miura et al. (Moll. Cell. Proteomics 7: 270-277 (2008)); Amano & Nishimura (Methods Enzymol. 478: 109-125 (2010)); and Aman et al. (ChemBioChem 13: 451-464 (2012)).

Materials and reagents for determining the N-glycan composition of a blood or blood component sample may be packaged in the form of a kit. Such kits typically will comprise a packaging material containing materials and reagents for performing the assay, such as at least one reagent for determining the N-glycan composition of a blood or blood component sample. The at least one reagent for determining the N-glycan composition of the blood or blood component sample may include a reagent for detecting one or more of a high mannose N-glycan, a hybrid N-glycan, a complex N-glycan, fucosylated N-glycan and/or an O-acetylated N-glycan, or combinations thereof. If the kit is for an immunoassay to determine the N-glycan composition of the blood or blood component sample, at least one of the reagents will be an antibody that specifically binds to the high mannose N-glycan, hybrid N-glycan, complex N-glycan, fucosylated N-glycan and/or O-acetylated N-glycan The N-glycan composition of the blood or blood component sample may then be used to determine a level of glycemic control or for diagnosing diabetes or pre-diabetes as disclosed herein. Such kits may optionally comprise instructions for determining the N-glycan composition using the at least one reagent. The instructions may further include guidance for interpreting the results of the assay.

By way of example, a kit for performing an immunoassay for monitoring the level of glycemic control according to the methods of the invention may contain, in a packaging material, at least one anti-glycan specific antibody targeting one or more of the N-glycans disclosed herein that is associated with an increase or decrease in glycemic control and, optionally, additional reagents, such as buffers or labeling reagents required for the immunoassay, and/or written instructions for performing the assay. Similarly, a kit for performing an immunoassay for diagnosing diabetes or pre-diabetes according to the methods of the invention may contain, in a packaging material, at least one anti-glycan specific antibody targeting one or more of the N-glycans disclosed herein that is associated with an increase or decrease in glycemic control and, optionally, additional reagents, such as buffers or labeling reagents required for the immunoassay, and/or written instructions for performing the assay. Such kits may further comprise reagents and/or materials for use as positive and/or negative assay controls.

### Example 1

### Methods

A 10 μl aliquot of each plasma sample was spiked with internal standard (700 pmol) and analyzed for N-linked glycans using Ezose Sciences' GLYCANMAP methodology. The samples were denatured and then digested with trypsin, followed by heat-inactivation. The mixture was then treated with PNGase F. After enzymatic release of N-glycans, aliquots were subjected to solid-phase processing using BLOTGLYCO beads. Following capture on the beads, the sialic acid residues were methyl esterified. The glycans were simultaneously released from the beads and labeled, and then aliquots of the recovered materials were spotted onto a MALDI target plate. Steps from initial aliquoting to spotting on the MALDI plate were performed using the fully automated SWEETBLOT technology. MALDI-TOF MS analysis was performed on an ultraflex III mass spectrometer (Bruker Daltonics) in the positive-ion, reflector mode. Each sample from the BLOTGLYCO bead processing step was spotted in quadruplicate, and spectra were obtained in an automated manner using the AutoXecute feature in flexControl software (Bruker Daltonics). Proposed glycan structures were assigned based on molecular weight. These methods have been described previously by Nishimura, Furukawa and Miura (Nishimura et al., Angew Chem. Int. Ed. Engl., 44: 91-96 (2004); Furukawa et al., Anal. Chem., 80: 1094-1101 (2008); Miura et al., Chem.-A Eur. J, 13: 4797-4804 (2007)). Alternatively, standard fluorescent HPLC or capillary electrophoresis methods with 2-AA, 2-AB, or APTS labeling can be used to monitor the glycan levels and changes in glycan patterns.

Rosigilitazone is a member of the athiazolidinedione class of anti-diabetic drugs, and is marketed by Glaxo under the trade name AVANDIA. Rosiglitazone works as an insulin sensitizer by binding to the peroxisome proliferator-activated receptors (PPAR) receptors in fat cells and making the cells more responsive to insulin. Diabetic (db/db) mice were treated once daily with an oral dose of 10 mpk rosiglitazone or with vehicle. Samples included plasma from 20 db/db mice (ten vehicle and ten rosiglitazone-treated) at each of seven time points: 3, 7, 10, 14, 21, 31, and 39 days. A baseline (Day 0) sample was not analyzed in the initial rosiglitazone study, but was included in a subsequent rosiglitazone confirmatory study. The confirmatory rosiglitazone study focused on glycan levels at baseline and 7 days.

Data Analysis:

Several criteria were used in the initial rosiglitazone study to select the most promising biomarkers. Statistical significance was evaluated based on the Mann-Whitney test or Student's t-test and changes considered significant if the resulting p-value was less than 0.05. Statistically significant differences were then compared across all available time points and only glycans that demonstrated statistically significant differences at 6 of the 7 time points and that exhibited changes that were sustained throughout the 39 day treatment period were selected. After the confirmatory rosiglitazone study, one glycan (530010) that had been excluded in the initial study was re-evaluated. This glycan was significant at 5 of 7 time points in the initial study and exhibited statistically significant differences at Day 7 in both rosiglitazone studies. It was therefore added to the original list of candidate biomarkers.

TABLE 1
Glycan Changes Associated with Glycemic Control
(Rosiglitazone Studies)

| | Direction of Change |
|---|---|
| Glycan Category and Code | with Rosiglitazone |
| High Mannose | |
| 520000 | Decreased |
| 620000 | Decreased |
| 720000 | Decreased |
| 820000 | Decreased |
| 920000 | Decreased |
| Fucosylated | |
| 651030 | Increased |
| 651031 | Increased |
| 761040 | Increased |
| O-Acetylated | |
| 540021 | Decreased |
| 540022 | Decreased |
| 540031 | Decreased |
| 540032 | Decreased |
| Hybrid | |
| 430010 | Decreased |
| 530010 | Decreased |
| 630010 | Decreased |
| Complex | |
| 540020 | Decreased |

### High-Mannose Glycans

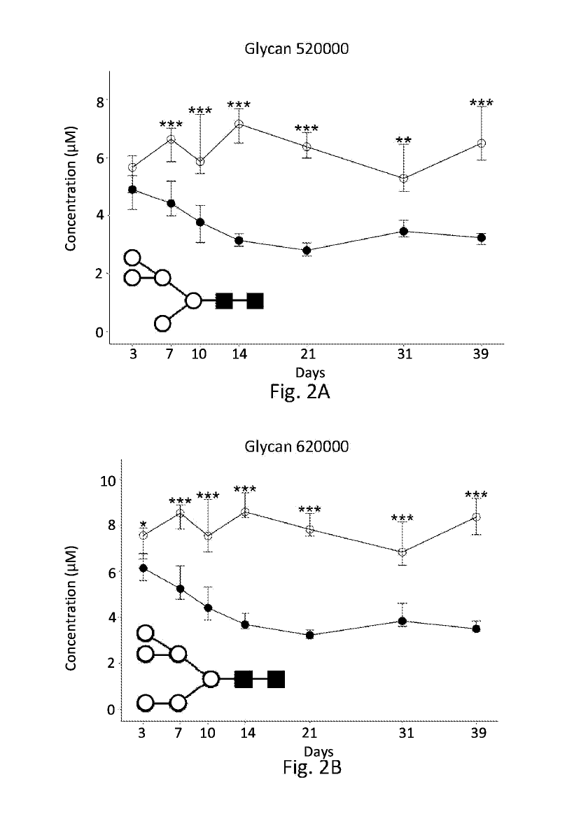

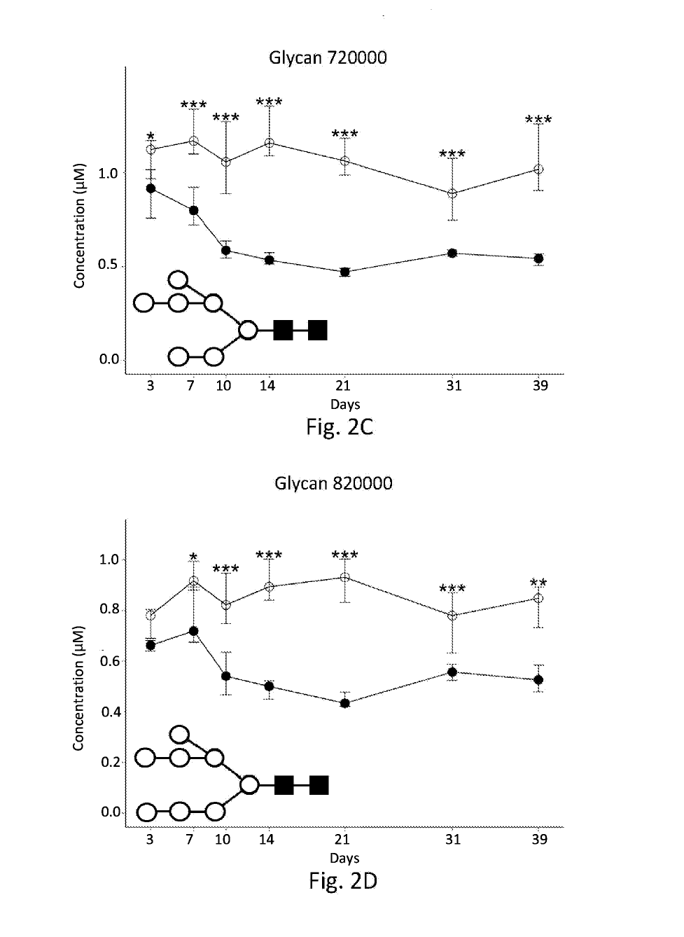

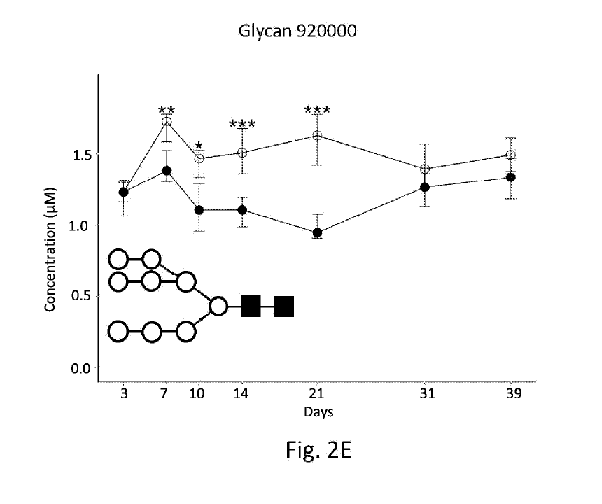

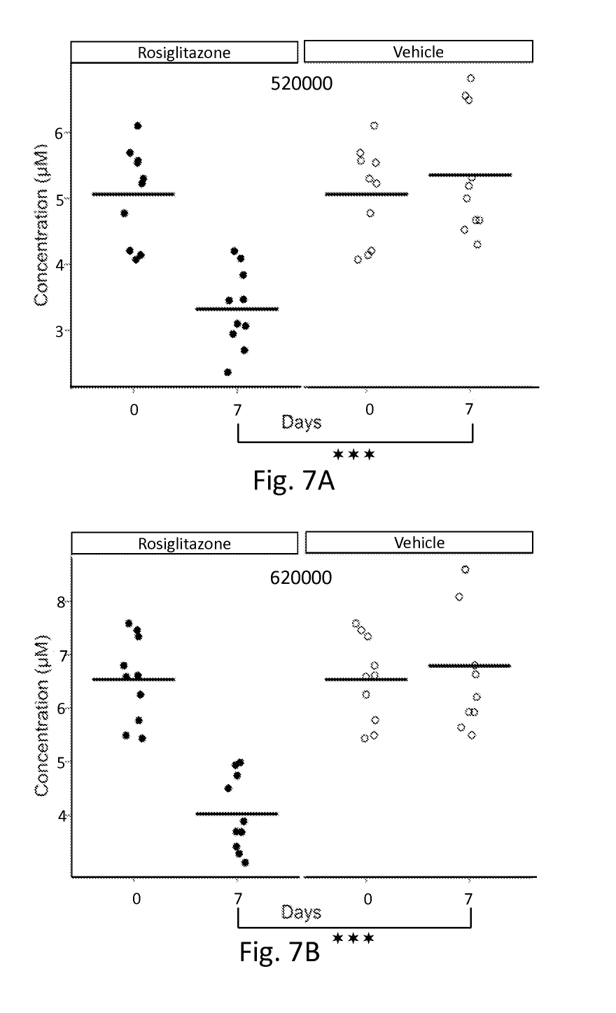

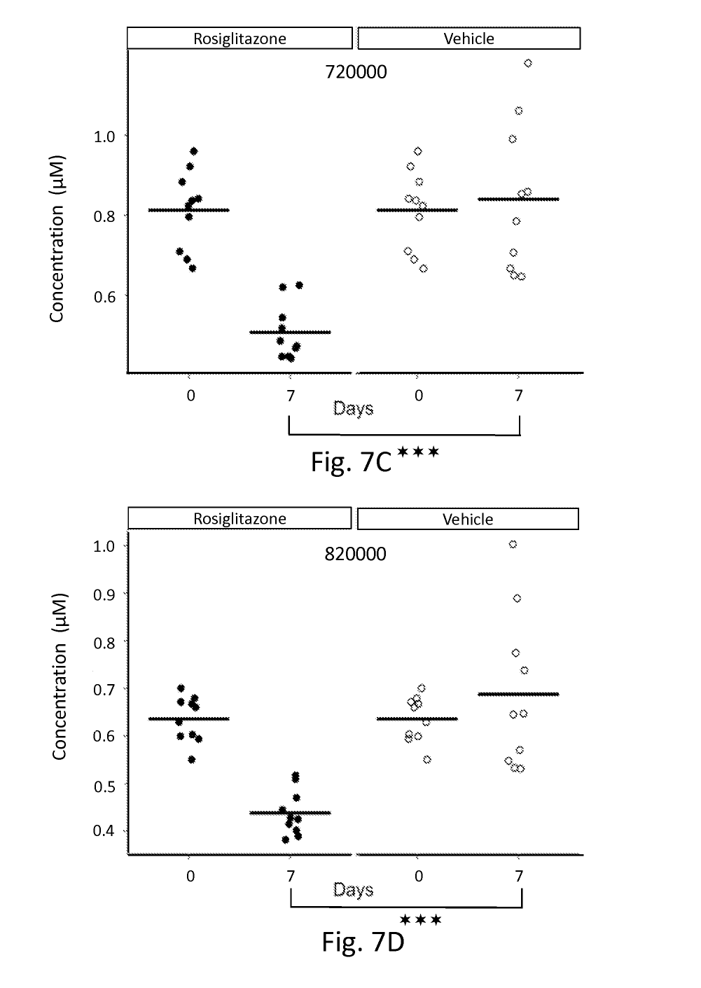

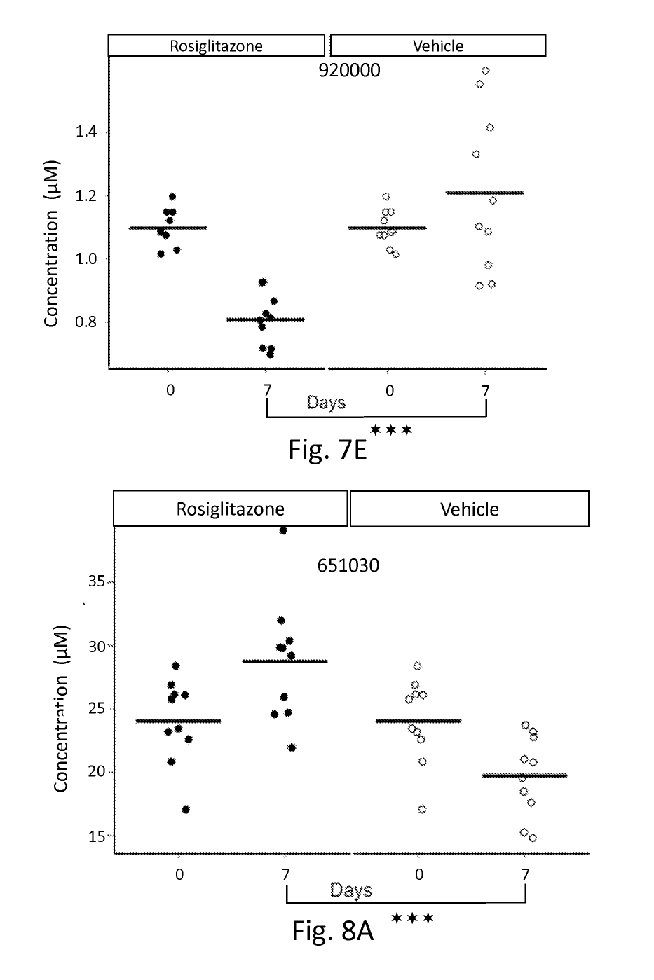

All five high-mannose N-glycans detected, including Man₅GlcNAc₂, Man₆GlcNAc₂, Man₇GlcNAc₂, Man₈GlcNAc₂, and Man₉GlcNAc₂ (glycan codes 520000, 620000, 720000, 820000, and 920000, respectively) were lower in rosiglitazone-treated db/db mice compared to vehicle-treated db/db mice in the first rosiglitazone study (FIGS. 2A-2E). Changes in all five high-mannose N-glycans were significant at Day 7, with two N-glycans (Man₆GlcNAc₂ and Man₇GlcNAc₂) exhibiting statistically significant differences between treatment groups at Day 3. Changes in all five high-mannose N-glycans were confirmed in a second rosiglitazone study (FIGS. 7A-7E).

### Fucosylated Glycans

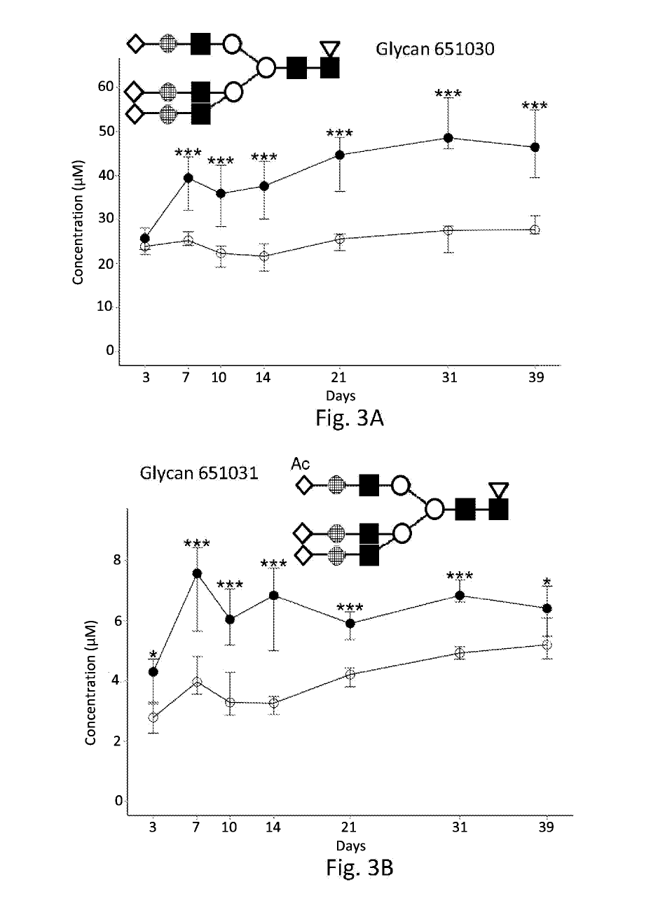

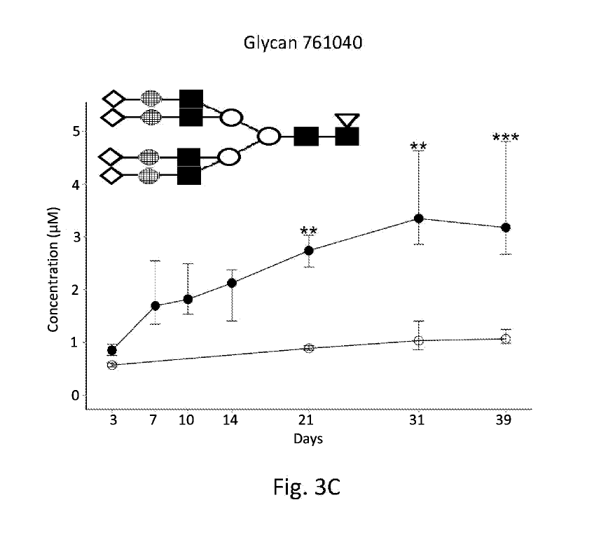

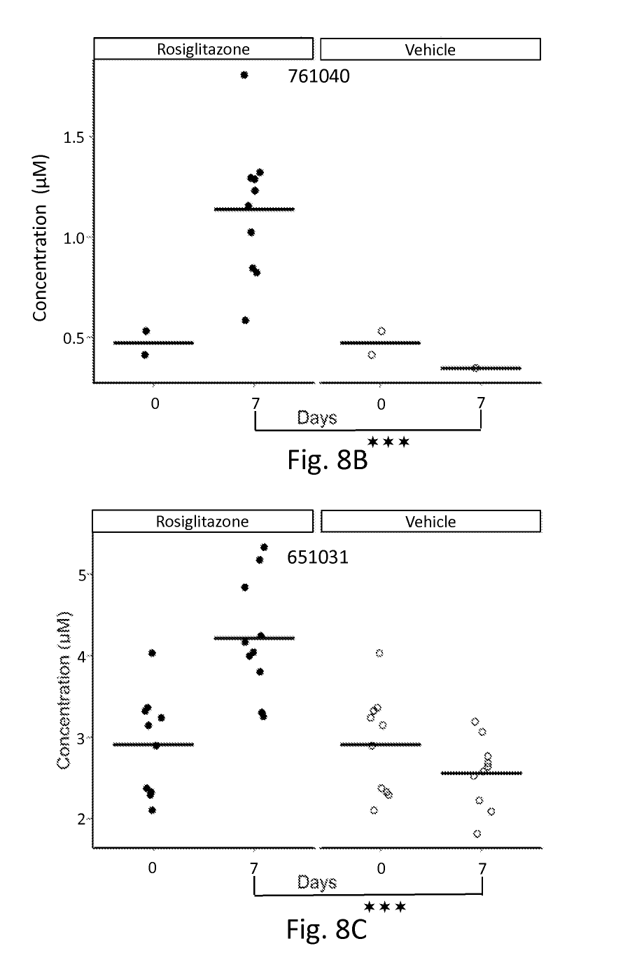

Several fucosylated glycans, including glycans 651030, 651031, and 761040 exhibited significantly higher levels in rosiglitazone-treated db/db mice compared to vehicle controls (FIGS. 3A-3C). Glycan 651030 and 651031 exhibited highly significant differences (p <0.001) at 7 days which were sustained at all subsequent time points analyzed in the first rosiglitazone study. Glycan 651031 also exhibited significant differences at Day 3. Changes in glycans 651030 and 651031 were confirmed in the second rosiglitazone study, which focused on changes at Day 7 (FIGS. 8A-8C). A third glycan (761040) showed a similar trend in both rosiglitazone studies but was lower abundance, making it difficult to quantitate in some samples.

### O-Acetylated Glycans

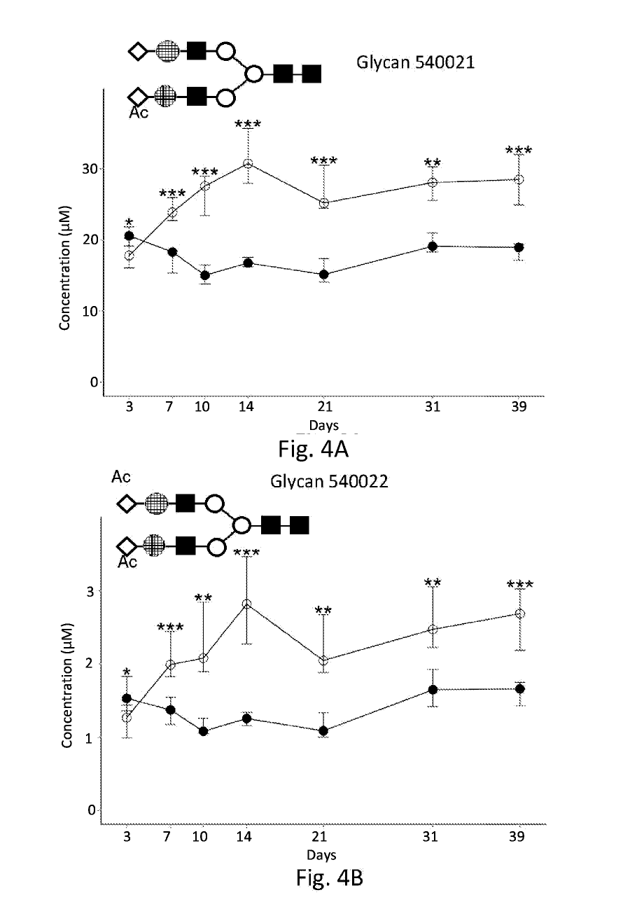

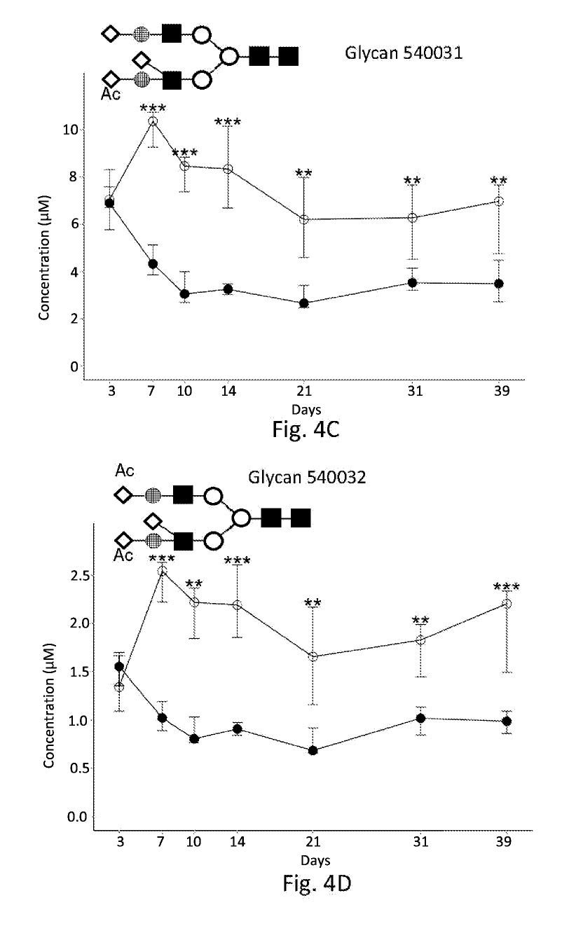

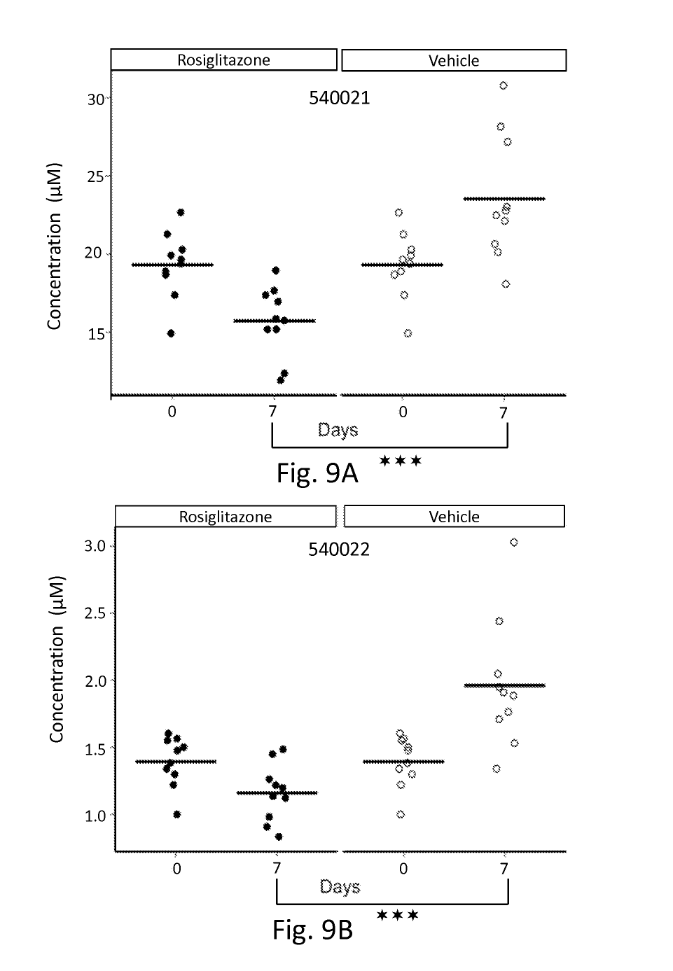

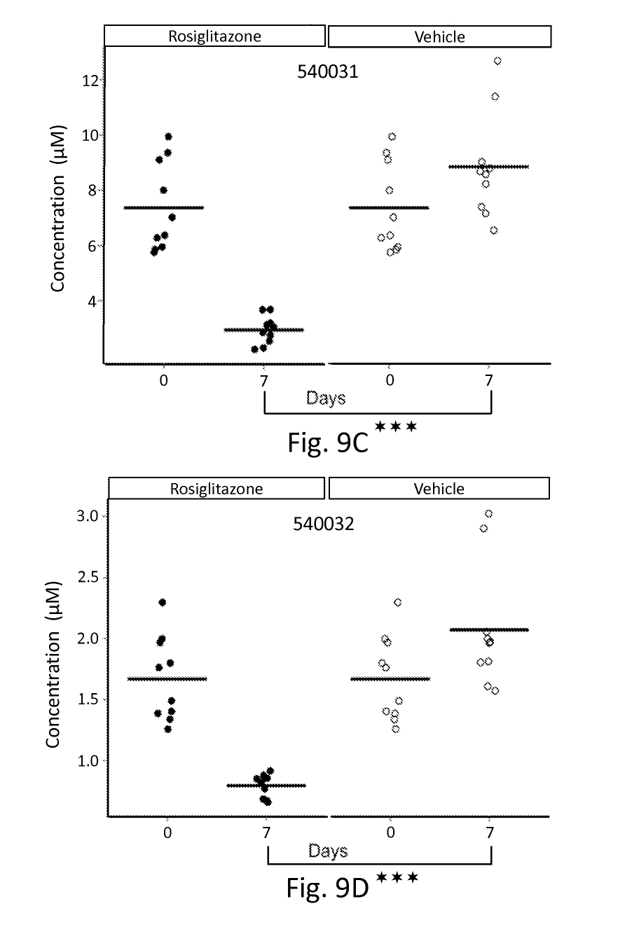

Acetylation of sialic acids in N-glycans is common in mice but is less common in humans. While acetylation of sialic acids has been reported in humans in cancerous cells, the presence and/or extent of O-acetylation in human diabetes is unknown. Several O-acetylated N-glycans exhibited statistically significant differences between treatment groups in both rosiglitazone studies. In the first study, four O-acetylated N-glycans, with glycan codes of 540021, 540022, 540031, and 540032 (FIGS. 4A-4D) exhibited significant lower levels (p <0.001) in rosiglitazone-treated db/db mice as early as seven days, which were sustained through the rest of the study. Glycans 540021 and 540022 showed significant differences as early as Day 3. Treatment-dependent differences in these N-glycans were confirmed in the second rosiglitazone study (FIGS. 9A-9D).

### Hybrid Glycans

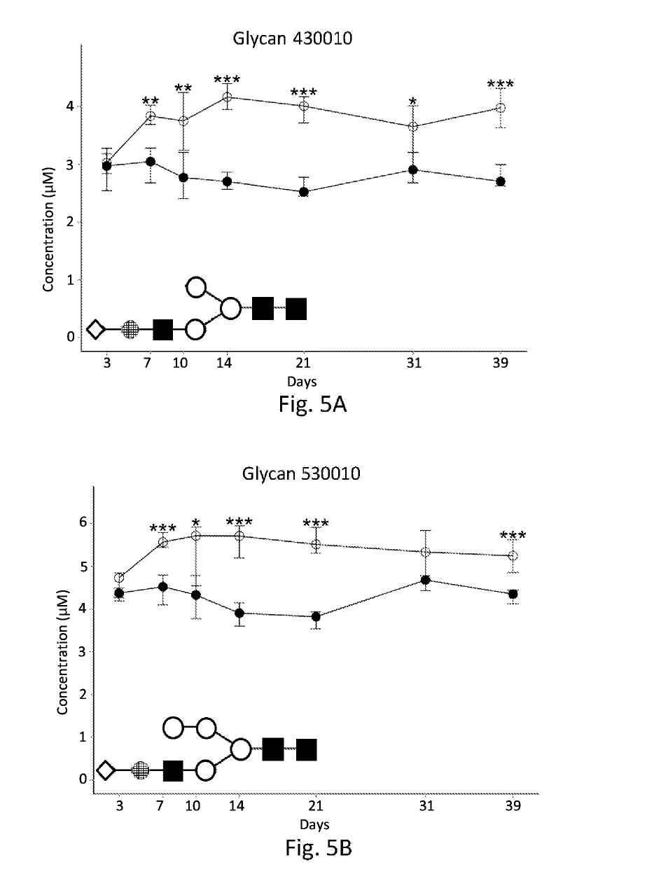

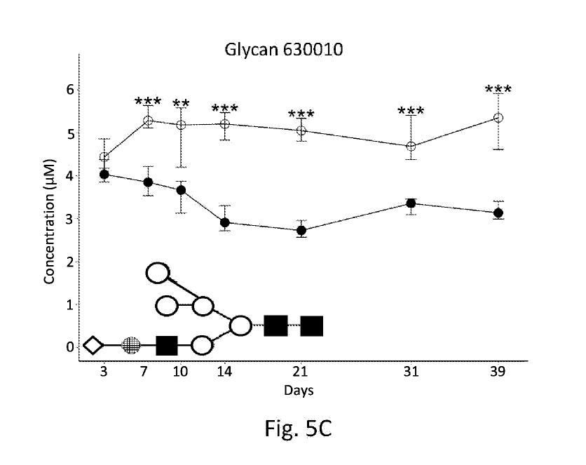

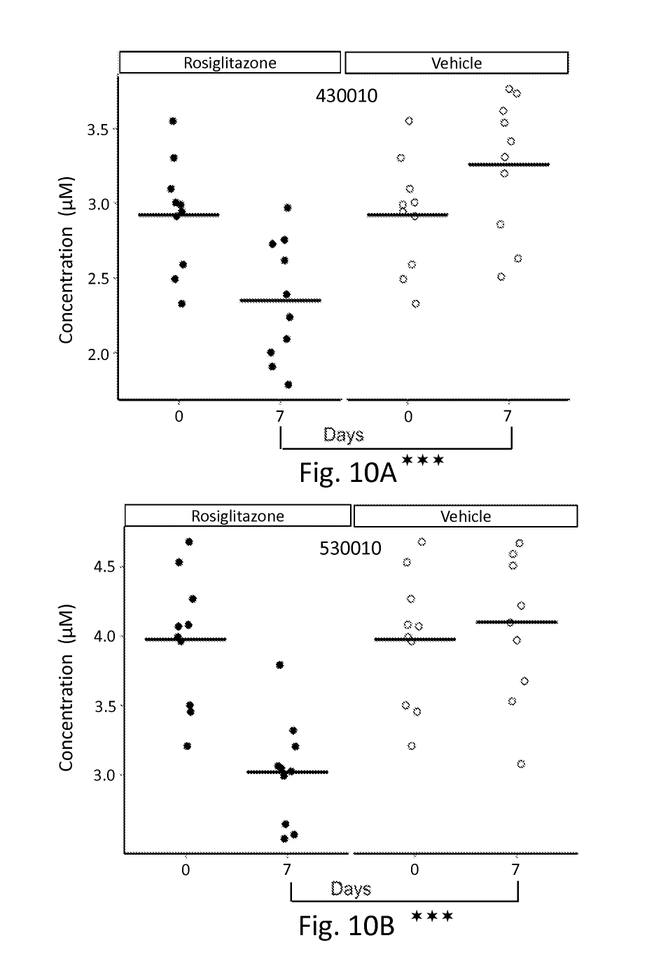

Three hybrid glycans (430010, 530010, and 630010) exhibited lower levels in rosiglitazone-treated db/db mice compared to the vehicle controls in the first rosiglitazone study (FIGS. 5A-5C). Changes in all three N-glycans were confirmed in the second rosiglitazone study, which demonstrated highly significant differences (p<0.001) at Day 7 (FIGS. 10A-10C).

### Complex Glycans

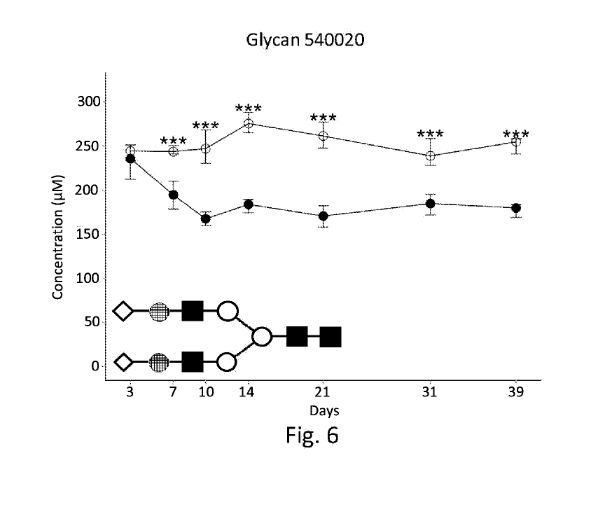

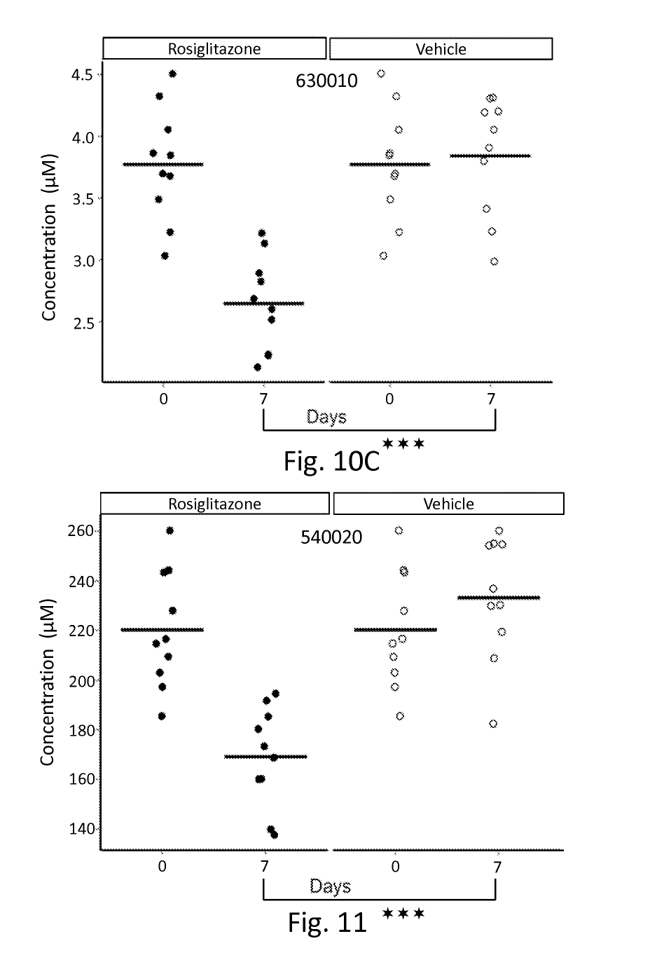

Complex glycan 540020 exhibited highly significant differences in rosiglitazone-treated mice compared to vehicle. In the first rosiglitazone study, Glycan 540020 exhibited a significant decrease in rosiglitazone-treated mice at Day 7 (p<0.001) which was sustained at subsequent time points (FIG. 6). This difference was confirmed in the second rosiglitazone study at Day 7 (p<0.0001) (FIG. 11).

Conclusions:

The rosiglitazone studies revealed statistically significant changes in 16 out of 52 individual N-glycans (Table 1). These glycan biomarkers could be grouped into several categories based on their structure. High-mannose, hybrid, O-acetylated, and complex glycans decreased with successful glycemic control, whereas fucosylated glycans increased. In the first rosiglitazone study, twelve of the 16 candidate biomarkers yielded highly significant differences (p-values <0.001) after seven days of treatment, with some glycans exhibiting significant differences after only 3 days. By comparison, this level of statistical significance was not achieved for HbA1c until 21 days, suggesting that changes in glycosylation on the circulating glycoproteins can predict subsequent changes in the level of glycation in HbA1c by approximately two weeks in this model. In the second rosiglitazone study, fifteen of the 16 candidate biomarkers yielded highly significant differences (p-values <0.001) after 7 days of treatment.

Reduction in high-mannose N-glycans was about a 2-3 week earlier leading indicator of the eventual reduction in HbA1c amounts that would be expected in a therapy or treatment regime that was effective for glycemic control. Fucosylated N-glycans were an about one week earlier indicator of the eventual reduction in amounts of HbA1c that would be expected in a therapy or treatment regime that was effective for glycemic control.

### Example 2

A further study was undertaken to evaluate the performance of candidate biomarkers discovered using rosiglitazone in mice treated with a diabetes drug having a different mechanism of action. The candidate biomarkers that were identified in Example 1 were evaluated in db/db mice treated with insulin detemir and vehicle.

Plasma samples were analyzed from ten db/db mice at baseline (0 days) and from 20 db/db mice (ten vehicle and ten insulin detemir-treated) at 7, 14, and 21 days. Sample preparation and analysis followed the protocol described in Example 1.

Data Analysis:

Concentrations of individual glycans in insulin detemir- and vehicle-treated db/db mice were compared at each time-point using the Student's t-test. N-glycans which yielded p-values <0.05 in this analysis were considered significant. Time-dependence was also evaluated for each of the candidate biomarkers by comparing each time-point to baseline. Results are summarized in Table 2:

TABLE 2
Glycan Changes Associated with Glycemic Control
(Insulin Detemir Study)

| | Direction of Change |
|---|---|
| Glycan Category and Code | with Insulin Detemir |
| High Mannose | |
| 520000 | Decreased |
| 620000 | Decreased |
| 720000 | Decreased |
| 820000 | Decreased |
| Hybrid | |
| 430010 | Decreased |
| 530010 | Decreased |
| 630010 | Decreased |

### High-Mannose Glycans

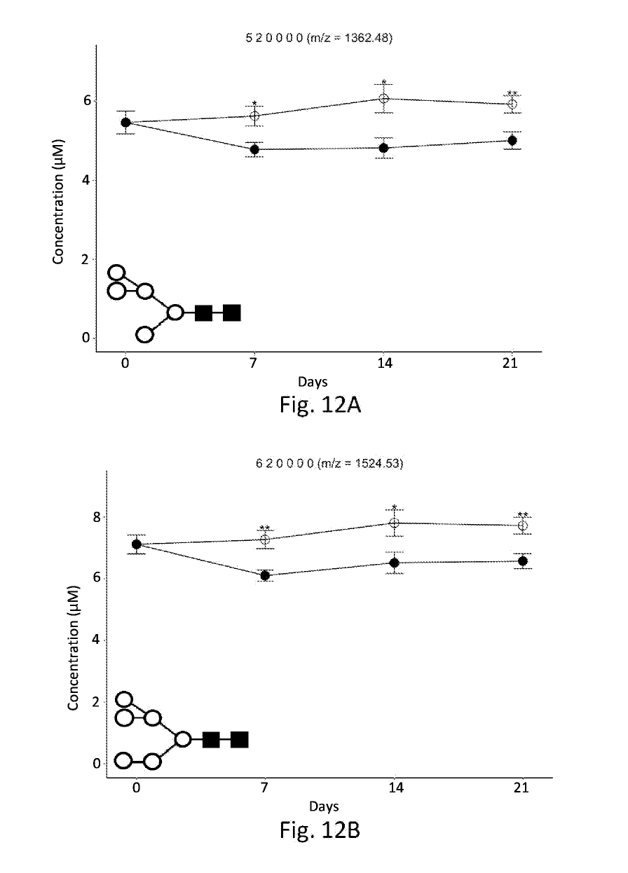

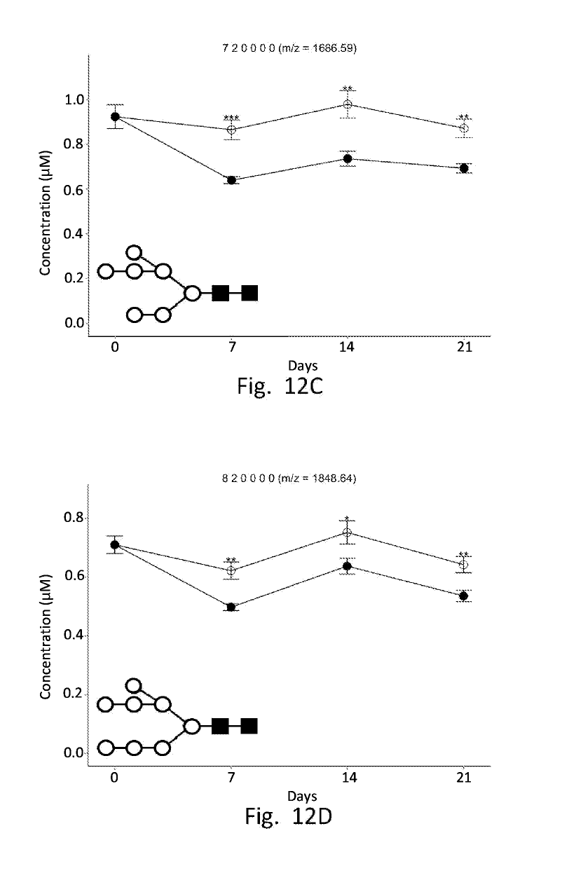

Insulin-induced changes in high-mannose glycans were lower in magnitude than with rosiglitazone, but four of the five high mannose glycans identified in the rosiglitazone studies also exhibited lower levels with insulin detemir treatment (Man₅GlcNAc₂ (520000), Man₆GlcNAc₂ (620000), Man₇GlcNAc₂ (720000), and Man₈GlcNAc₂ (820000)). The differences were significant at Day 7 and remained significant at Day 14 and 21 (FIGS. 12A-12D).

### Hybrid Glycans

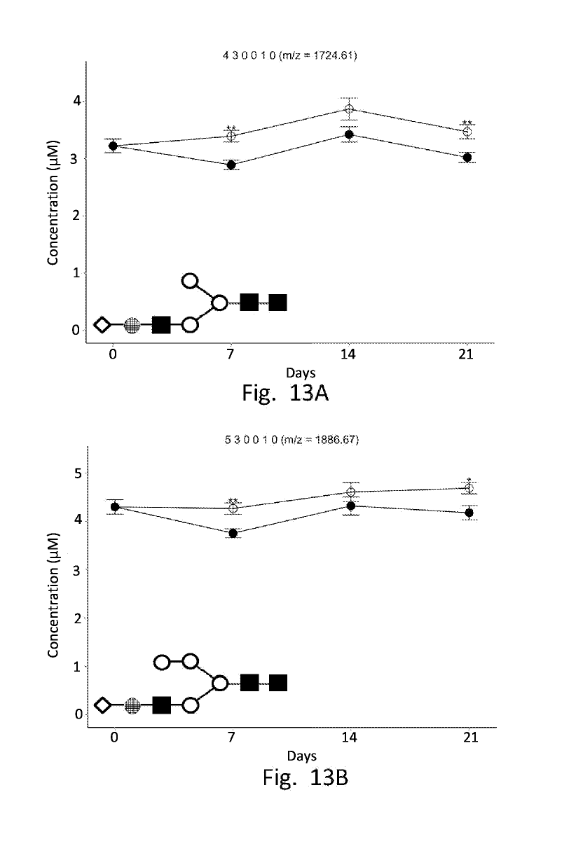

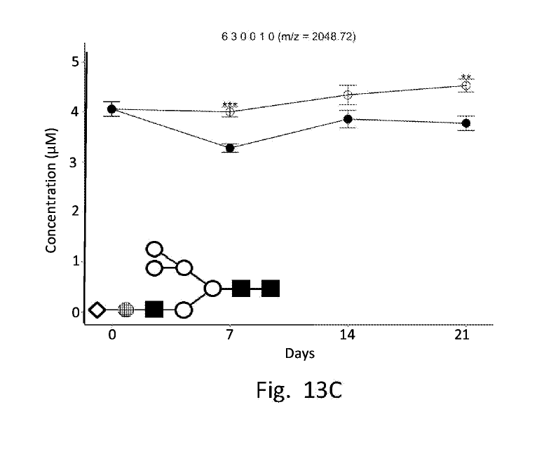

Three hybrid glycans, SiaGalGlcNAcMan₃GlcNAc₂ (430010), SiaGalGlcNAcMan₄GlcNAc₂ (530010), and SiaGalGlcNAcMan₅GlcNAc₂ (630010), demonstrated statistically significant differences between insulin detemir-treated db/db mice and the vehicle-treated controls (FIGS. 13A-13C). These glycans were also lower in rosiglitazone-treated mice. All three hybrid glycans showed significant decreases in insulin detemir-treated mice as early as Day 7.

In contrast to treatment with rosiglitazone, no significant changes in fucosylated N-glycans, O-acetylated N-glycans, or Glycan 540020 were observed in mice treated with insulin detemir.

Conclusions:

Both high-mannose and hybrid N-glycans decreased as early as 7 days after initiation of treatment in rosiglitazone- and insulin detemir-treated db/db mice, suggesting that both drugs induce similar changes and that these glycans, in particular, are indicative of glycemic control. The db/db mice treated with rosiglitazone exhibited increasing separation from the vehicle-treated db/db mice over time. While separation between treatment groups was less pronounced in insulin-treated db/db mice, this result is consistent with the more subtle changes in HbA1c that were observed. Nevertheless, a shift in the same direction as rosiglitazone-treated mice was observed, reinforcing the similarity of changes induced by rosiglitazone and insulin detemir.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. A method of monitoring a level of glycemic control in a subject during anti-diabetic therapy or treatment comprising:
(a) providing an N-glycan composition of a blood or blood component sample obtained from the subject at a first time-point during the anti-diabetic therapy or treatment; and
(b) determining an N-glycan composition of a blood or blood component sample obtained from the subject at a second time-point during the anti-diabetic therapy or treatment, wherein the second time-point is subsequent to the first time-point,
wherein a difference between the N-glycan composition of the sample at the second time-point and the N-glycan composition of the sample at the first time-point indicates an increased or decreased level of glycemic control at the second time-point compared to the first time-point.

2. The method of claim 1, wherein an increased level of glycemic control at the second time-point compared to the first time-point is indicated by
i) a decrease in an amount of at least one high mannose N-glycan, hybrid N-glycan, complex N-glycan, and/or O-acetylated N-glycan in the N-glycan composition of the blood or blood component sample at the second time-point as compared to an amount of a corresponding N-glycan in the N-glycan composition of the blood or blood component sample at the first time-point, and/or;
ii) an increase in an amount of one or more fucosylated N-glycans in the N-glycan composition of the blood or blood component sample at the second time-point as compared to an amount of a corresponding fucosylated N-glycan in the N-glycan composition of the blood or blood component sample at the first time-point.

3. The method of claim 2, wherein the at least one high mannose N-glycan is selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000).

4. The method of claim 2, wherein the at least one hybrid N-glycan is selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

5. The method of claim 2, wherein the at least one complex N-glycan is Sia₂Gal₂GlcNAc₂Man₃GlcNAc₂ (540020), wherein Sia is Neu5Ac or Neu5Gc.

6. The method of claim 2, wherein the at least one O-acetylated (O-Ac) N-glycan is selected from the group consisting of Sia₂Gal₂GlcNAc₂Man₃GlcNAc₂ (1 O-Ac) (540021), Sia₂Gal₂GlcNAc₂Man₃GlcNAc₂ (2 O-Ac) (540022), Sia₃Gal₂GlcNAc₂Man₃GlcNAc₂ (1 O-Ac) (540031), and Sia₃Gal₂GlcNAc₂Man₃GlcNAc₂ (2 O-Ac) (540032), wherein Sia is Neu5Ac or Neu5Gc.

7. The method of claim 2, wherein the at least one fucosylated N-glycan is selected from the group consisting of Sia₃Gal₃GlcNAc₃Man₃GlcNAc₂ (Fuc) (651030), Sia₃Gal₃GlcNAc₃Man₃GlcNAc₂ (Fuc) (1 O-Ac) (651031), and Sia₄Gal₄GlcNAc₄Man₃GlcNAc₂ (Fuc) (761040), wherein Sia is Neu5Ac or Neu5Gc.

8. The method of claim 2, wherein amounts of N-glycans are determined using MALDI-TOF, HPLC, capillary electrophoresis or immunoassay.

9. The method of claim 1, wherein the difference in N-glycan composition between the second and first time-points is a statistically significant increase or decrease in an amount of at least one N-glycan.

10. The method of claim 1, wherein a decrease in at least one high-mannose N-glycan and/or a decrease in at least one hybrid N-glycan indicates an increased level of glycemic control at the second time-point compared to the first time-point.

11. The method of claim 10, wherein the at least one high-mannose N-glycan is selected from the group consisting of Man₅GlcNAc₂ (520000), Man₆GlcNAc₂ (620000), Man₇GlcNAc₂ (720000), Man₈GlcNAc₂ (820000) and Man₉GlcNAc₂ (920000), and/or the at least one hybrid N-glycan is selected from the group consisting of SiaGalGlcNAcMan₃GlcNAc₂ (430010), SiaGalGlcNAcMan₄GlcNAc₂ (530010) and SiaGalGlcNAcMan₅GlcNAc₂ (630010).

12. A method of diagnosing diabetes mellitus or pre-diabetes in a subject comprising:
(a) determining an N-glycan composition of a blood or blood component sample obtained from the subject; and
(b) comparing the N-glycan composition of the blood or blood component sample of the subject to an N-glycan composition of a normoglycemic blood or blood component,
wherein a difference between the N-glycan composition of the blood or blood component sample from the subject and the N-glycan composition of the normoglycemic blood or blood component indicates diabetes mellitus or pre-diabetes in the subject.

13. The method of claim 12, wherein diabetes mellitus or pre-diabetes is indicated by
i) an increase in an amount of at least one high mannose N-glycan, hybrid N-glycan, complex N-glycan, and/or O-acetylated N-glycan in the N-glycan composition of the blood or blood component sample of the subject as compared to an amount of a corresponding N-glycan in the N-glycan composition of the normoglycemic blood or blood component, and/or;
ii) a decrease in an amount of one or more fucosylated N-glycans in the N-glycan composition of the blood or blood component sample of the subject as compared to an amount of a corresponding fucosylated N-glycan in the N-glycan composition of the normoglycemic blood or blood component.

14. The method of claim 13, wherein the at least one high mannose N-glycan is selected from the group consisting of Man₉GlcNAc₂ (920000), Man₈GlcNAc₂ (820000), Man₇GlcNAc₂ (720000), Man₆GlcNAc₂ (620000), and Man₅GlcNAc₂ (520000).

15. The method of claim 13, wherein the at least one hybrid N-glycan is selected from the group consisting of SiaGalGlcNAcMan₃GlcNAc₂ (430010), SiaGalGlcNAcMan₄GlcNAc₂ (530010), and SiaGalGlcNAcMan₅GlcNAc₂ (630010), wherein Sia is Neu5Ac or Neu5Gc.

16. The method of claim 13, wherein the at least one complex N-glycan is Sia₂Gal₂GlcNAc₂Man₃GlcNAc₂ (540020), wherein Sia is Neu5Ac or Neu5Gc.

17. The method of claim 2, wherein the at least one O-acetylated (O-Ac) N-glycan is selected from the group consisting of Sia₂Gal₂GlcNAc₂Man₃GlcNAc₂ (1 O-Ac) (540021), Sia₂Gal₂GlcNAc₂Man₃GlcNAc₂ (2 O-Ac) (540022), Sia₃Gal₂GlcNAc₂Man₃GlcNAc₂ (1 O-Ac) (540031), and Sia₃Gal₂GlcNAc₂Man₃GlcNAc₂ (2 O-Ac) (540032), wherein Sia is Neu5Ac or Neu5Gc.

18. The method of claim 13, wherein the at least one fucosylated N-glycan is selected from the group consisting of Sia₃Gal₃GlcNAc₃Man₃GlcNAc₂ (Fuc) (651030), Sia₃Gal₃GlcNAc₃Man₃GlcNAc₂ (Fuc) (1 O-Ac) (651031), and Sia₄Gal₄GlcNAc₄Man₃GlcNAc₂ (Fuc) (761040), wherein Sia is Neu5Ac or Neu5Gc.

19. The method of claim 13, wherein amounts of N-glycans are determined using MALDI-TOF, HPLC, capillary electrophoresis or immunoassay.

20. The method of claim 12, wherein the difference in N-glycan composition between the subject blood or blood component sample and the normoglycemic blood or blood component is a statistically significant increase or decrease in an amount of at least one N-glycan.

21. The method of claim 12, wherein an increase in at least one high-mannose N-glycan and/or an increase in at least one hybrid N-glycan indicates a diagnosis of diabetes or pre-diabetes.

22. The method of claim 21, wherein the at least one high-mannose N-glycan is selected from the group consisting of Man₅GlcNAc₂ (520000), Man₆GlcNAc₂ (620000), Man₇GlcNAc₂ (720000), Man₈GlcNAc₂ (820000) and Man₉GlcNAc₂ (920000), and/or the at least one hybrid N-glycan is selected from the group consisting of SiaGalGlcNAcMan₃GlcNAc₂ (430010), SiaGalGlcNAcMan₄GlcNAc₂ (530010) and SiaGalGlcNAcMan₅GlcNAc₂ (630010).

23. The method of claim 12, wherein the N-glycan composition of the blood or blood component sample of the subject is compared to the N-glycan composition of a normoglycemic blood or blood component sample previously obtained from the subject.

24. A biomarker for monitoring a level of glycemic control during anti-diabetic therapy or treatment, or for diagnosing diabetes mellitus, which comprises an N-glycan profile of blood or blood component proteins.

25.-27. (canceled)

28. A kit for monitoring a level of glycemic control during anti-diabetic therapy or treatment, or for diagnosing diabetes mellitus, which comprises:
a) a packaging material containing the biomarker according to claim 24 and at least one reagent for determining an N-glycan composition of blood or a blood component sample, wherein the N-glycan composition comprises one or more of a high mannose N-glycan, a hybrid N-glycan, a complex N-glycan, a fucosylated N-glycan and/or an O-acetylated N-glycan, or combinations thereof, and
b) optionally, instructions for determining the N-glycan composition using the at least one reagent.

29. The kit of claim 28, further comprising reagents and/or materials for use as positive or negative controls.

30.-31. (canceled)
